(19) United States
(12) Patent Application Publication
(10) Pub. No.: US 2003/0109842 A1
(43) Pub. Date: Jun. 12, 2003

(54) Separated targeted elastic zone for improved process and product function

(76) Inventors: Raymond Gerard St. Louis, Fremont, WI (US); James Marcus Carr, Kaukauna, WI (US); Adrian Roy Eggen, Appleton, WI (US); Howard Martin Welch, Woodstock, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/015,935

(22) Filed: Dec. 12, 2001

(51) Int. Cl. C09J 5/00; B65C 9/25; A61F 13/15; A61F 13/20

(52) U.S. Cl. 604/385.29; 604/385.27; 156/324

(57) ABSTRACT

A disposable garment includes a chassis defining openings for the legs and waist of a wearer and side panels. The side panels include a targeted elastic material including zones of high and low elastic tension in the same material, integrated during formation of the material. The targeted elastic material is positioned so that at least one high tension zone is aligned with the waist opening, thereby functioning as an elastic band without requiring a separately manufactured, attached elastic band. A spacer zone, comprising a low tension zone or a no tension zone, is positioned between the at least one high tension zone, which is aligned with the waist opening, and the waist end edge of the side panel.

### BACKGROUND OF THE INVENTION

This invention relates generally to a garment having an integrated zone of elastic tension, and more particularly to a garment having an integrated zone of elastic tension and a spacer zone, including a zone of low elastic tension or no elastic tension, between the integrated zone of elastic tension and an edge of the garment.

Garments, including absorbent garments, medical garments, and other products, are commonly made with an elastic band adjacent at least one of the garment edges. A pant-like garment, for instance, may have an elastic band adjacent the waist opening, each of the two leg openings, or all three of the openings. The elastic band adjacent the waist opening holds the garment in place, and prevents it from falling off of the wearer. The elastic bands adjacent the leg openings help to seal the garment against the wearer's legs, thereby preventing or reducing leakage of waste materials from inside the garment.

In conventional garments, the primary material for the garment is manufactured and assembled separately from the elastic bands. Following their separate manufacture, the elastic bands are attached to the primary material at some stage during manufacture of the garment by sewing, ultrasonic welding, thermal bonding, adhesive bonding, or the like. In the resulting product, the user can often see the elastic band as a distinct entity attached to the garment.

The use of elastic side panels is known in the art. Suitable elastic materials, as well as one described process of incorporating elastic side panels into a training pant, are described in the following U.S. Pat. No. 4,940,464 issued Jul. 10, 1990 to Van Gompel et al.; U.S. Pat. Nos. 5,224,405 issued Jul. 6, 1993 to Pohjola; 5,104,116 issued Apr. 14, 1992 to Pohjola; and U.S. Pat. No. 5,046,272 issued Sep. 10, 1991 to Vogt et al.; all of which are incorporated herein by reference.

U.S. Pat. No. 5,674,216 issued Oct. 7, 1997 to Buell et al. describes elasticized side panels which may exhibit differential extensibility along the longitudinal axis of the side panel when stretched in a lateral direction. However, Buell et al. does not describe distinct zones of high and low elastic tension in the side panels. Furthermore, Buell et al. discloses passing a laminate through corrugated rolls having corrugations of nonuniform profile along points of contact with the laminate.

There is a need or desire to reduce both material and manufacturing costs associated with garments and to minimize process variability, without sacrificing performance and quality. However, this should be accomplished without compromising the performance characteristics of the various regions in the garment.

### SUMMARY OF THE INVENTION

The present invention is directed to a garment having one or more garment edges. The garment has elastic properties at the edges achieved without the use of a separately manufactured, separately attached elastic band, and is easier and less expensive to manufacture than a conventional garment having one or more elastic bands at or near the edges.

In one aspect of the invention, side panels of a garment are manufactured using a targeted elastic material (“TEM”) having a targeted elastic zone aligned with the waist opening and a spacer zone, including a zone of low tension or no tension, between the targeted elastic zone and the waist end edge of each side panel. The TEM can have a substantially homogeneous appearance, and does not need a separately manufactured elastic band attached to it. Yet the TEM has different elastic properties at different regions, and exhibits greater elastic tension in a region aligned with the waist opening. Furthermore, in particular embodiments the TEM abuts a garment edge or opening.

During manufacturing of the garments, a first zone, which corresponds to a double “spacer zone” on the side panel strip, is cut in two. The first zone is located between targeted elastic second zones on consecutive uncut garment side panels so that cutting the first zone in two along a cut line provides a single spacer zone between the targeted elastic zone and the waist end edge of the side panel of each resulting individual garment. In this way, process variability in the manufacture of the garment is reduced. By leaving the spacer zone between the targeted elastic zone and the cut line on consecutive garment side panels, the size of the targeted elastic zone can be manufactured consistently. Without the spacer zone, a double-sized targeted elastic zone in the side panels of the consecutive uncut garments would be cut in two possibly resulting in the leading garment having a longer targeted elastic zone and the trailing garment having a shorter targeted elastic zone, or vice versa.

In addition, the presence of the spacer zone minimizes any waste that may be associated with the targeted elastic zone extending all the way to the cut edge of the side panel. Furthermore, spacing the targeted elastic zone down from the top edge of the side panel may have a positive impact on the fit of the garments and may assist in applying the garment to the wearer.

The identified problem of variability in the cutting process which may yield elastic regions with inconsistent dimensions applies not only to elastic regions in training pants, as previously mentioned, but also to elastic regions in any other type of personal care garment in which an integrated (or non-integrated) zone of elastic tension is aligned with an edge of the garment.

With the foregoing in mind, it is a feature and advantage of this invention to provide a garment including a targeted elastic material with a spacer zone between the targeted elastic zone and an edge of the garment.

These and other features and advantages will become further apparent from the following detailed description of the presently preferred embodiments, read in conjunction with the drawings.

### BRIEF DESCRIPTION OF THE DRAWINGS

FIG. 1 illustrates a perspective view of a pant-like absorbent garment in accordance with the invention, having targeted elastic material in the side panels;

FIG. 2 illustrates another embodiment of a pant-like absorbent garment of the invention;

FIG. 3 is a plan view of the garment shown in FIG. 1, showing the side facing away from the wearer;

FIG. 4 is a plan view of the garment shown in FIG. 1, showing the side facing the wearer;

FIGS. 5-8 illustrate representative targeted elastic laminate (“TEL”) materials useful for making the garments of FIGS. 1 and 2;

FIGS. 9-12 illustrate representative processes for making TEL materials useful for making garments of the invention;

FIG. 13A shows one exemplary adhesive spray pattern in which the adhesive has been applied to the elastic filaments with attenuation in the cross direction;

FIG. 13B shows a second exemplary adhesive spray pattern;

FIG. 13C illustrates a third exemplary adhesive spray pattern;

FIG. 13D shows an exemplary bond angle in one exemplary adhesive spray pattern;

FIG. 13E shows one exemplary adhesive pattern in which the adhesive has also been applied in the no tension zone including no elastic filaments;

FIG. 14 illustrates the bonding pattern and method of calculating the number of bonds per unit length on elastic strands or filaments;

FIG. 15A shows a fourth exemplary adhesive spray pattern in a swirled-type of configuration;

FIG. 15B shows a fifth exemplary adhesive spray pattern that is more randomized and which provides a large percentage of adhesive lines in a perpendicular orientation to the elastic filaments;

FIG. 15C illustrates a sixth exemplary adhesive spray pattern having attenuation of adhesive lines in the cross-machine direction;

FIG. 15D shows a seventh exemplary adhesive spray pattern that resembles a “chain-link fence”;

FIG. 16 is a schematic view of another process for making TEL materials useful for making garments of the invention;

FIG. 17A is a plan view of a machine direction product assembly for producing an absorbent garment with targeted elastic material in the side panels without a spacer zone between the targeted elastic zone and the waist end edge of each side panel;

FIG. 17B is a plan view of a machine direction product assembly for producing an absorbent garment with targeted elastic material in the side panels with a spacer zone between the targeted elastic zone and the waist end edge of each side panel;

FIGS. 18A-18C are plan views of an assembly section for making a continuous stream of partially assembled discrete training pants;

FIG. 19 is a plan view of a face mask made of the targeted elastic material of the present invention; and

FIGS. 20A-20B illustrate representative TEL materials useful for making the face mask of FIG. 19.

### DEFINITIONS

The term “elastic band” refers to a discrete elongated element having elastic properties. The term “discrete elongated element” refers to a long, relatively narrow element that is separately manufactured and then attached to an underlying material, and does not include elongated regions having elastic properties that are part of an underlying material as made. The terms “elastic” and “elastomeric” are used interchangeably to mean a material that is generally capable of recovering its shape after deformation when the deforming force is removed. Specifically, as used herein, elastic or elastomeric is meant to be that property of any material which upon application of a biasing force, permits that material to be stretchable to a stretched biased length which is at least about 50 percent greater than its relaxed unbiased length, and that will cause the material to recover at least 40 percent of its elongation upon release of the stretching force. A hypothetical example which would satisfy this definition of an elastomeric material would be a one (1) inch sample of a material which is elongatable to at least 1.50 inches and which, upon being elongated to 1.50 inches and released, will recover to a length of not more than 1.30 inches. Many elastic materials may be stretched by much more than 50 percent of their relaxed length, and many of these will recover to substantially their original relaxed length upon release of the stretching force.

The term “inelastic” refers to materials that are not elastic.

The term “targeted elastic regions” or “targeted elastic zones” refers to isolated, often relatively narrow regions or zones in a single composite material or layer, which have greater elastic tension than adjacent or surrounding regions or zones.

The term “targeted elastic material” (“TEM”) refers to a single elastic material or laminate having targeted elastic zones. TEM's include only materials or laminates which are made in a single manufacturing process, and which are capable of exhibiting targeted elastic properties without requiring an added elastic band or layer in the targeted elastic region. TEM's do not include materials having elasticized regions achieved through separate manufacture of an elastic band, and subsequent connection of the elastic band to the underlying material.

The term “targeted elastic laminate” or “TEL” refers to an elastic laminate which behaves as a TEM. The TEL suitably includes at least one elastic nonwoven filament web, in which different zones of different elastic tension exist across a width of the web when the laminate is stretched in a longitudinal direction perpendicular to the width. The different zones may, but do not necessarily, have different elongations at break, or recoveries. What is important is that the different zones exhibit different levels of retractive force when the laminate is uniformly stretched by a selected amount. The elastic nonwoven filament web is laminated to at least one other layer, whereby the laminate exhibits different levels of elastic tension in zones corresponding to the high and low tension zones in the nonwoven filament web.

The term “targeted elastic stretch-bonded laminate” or “TE SBL” refers to a TEL which is formed by stretching an elastic nonwoven filament web having zones of different elastic tension, maintaining the stretched condition of the elastic nonwoven filament web when the other layer is bonded to it, and relaxing the TEL after bonding.

The term “vertical filament stretch-bonded laminate” or “VF SBL” refers to a stretch-bonded laminate made using a continuous vertical filament process, as described herein.

The term “continuous filament stretch-bonded laminate” or “CF SBL” refers to a stretch-bonded laminate made using a continuous horizontal filament process, as described herein.

The term “elastic tension” refers to the amount of force per unit width required to stretch an elastic material (or a selected zone thereof) to a given percent elongation.

The term “low tension zone” or “lower tension zone” refers to a zone or region in a stretch-bonded laminate material having one or more filaments with low elastic tension characteristics relative to the filament(s) of a high tension zone, when a stretching or biasing force is applied to the stretch-bonded laminate material. Thus, when a biasing force is applied to the material, the low tension zone will stretch more easily than the high tension zone. At 50% elongation of the fabric, the high tension zone may exhibit elastic tension at least 10% greater, suitably at least 50% greater, desirably about 100-800% greater, or alternatively about 150-300% greater than the low tension zone.

The term “high tension zone” or “higher tension zone” refers to a zone or region in a stretch-bonded laminate material having one or more filaments with high elastic tension characteristics relative to the filament(s) of a low tension zone, when a stretching or biasing force is applied to the stretch-bonded laminate material. Thus, when a biasing force is applied to the material, the high tension zone will stretch less easily than the low tension zone. Thus, high tension zones have a higher tension than low tension zones. The terms “high tension zone” and “low tension zone” are relative, and the material can have multiple zones of different tensions.

The term “no tension zone” refers to a zone or region in a stretch-bonded laminate material having no filaments, the zone having very low or no elastic tension characteristics relative to the low or high tension zones, when a stretching or biasing force is applied to the stretch-bonded laminate material.

The term “spacer zone” refers to a zone or region of a TEM within a garment. The spacer zone has low tension, or no tension.

The term “nonwoven fabric or web” means a web having a structure of individual fibers or filaments which are interlaid, but not manipulated in a manner such as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The term also includes films that have been cut into narrow strips, perforated or otherwise treated to allow air to pass through. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Liquid-impermeable,” when used to describe a layer or laminate means that liquid such as urine will not pass through the layer or laminate under ordinary use conditions in a direction generally perpendicular to the plane of the layer or laminate at the point of liquid contact.

“Liquid-permeable,” refers to a layer or laminate that is not liquid impermeable.

The term “microfibers” means small diameter fibers having an average diameter not greater than about 75 microns, for example, having an average diameter of from about 1 micron to about 50 microns, or more particularly, having an average diameter of from about 1 micron to about 30 microns.

The term “machine direction” refers to the length of a fabric in the direction in which it is produced, as opposed to “cross-machine direction” which refers to the width of a fabric in a direction generally perpendicular to the machine direction.

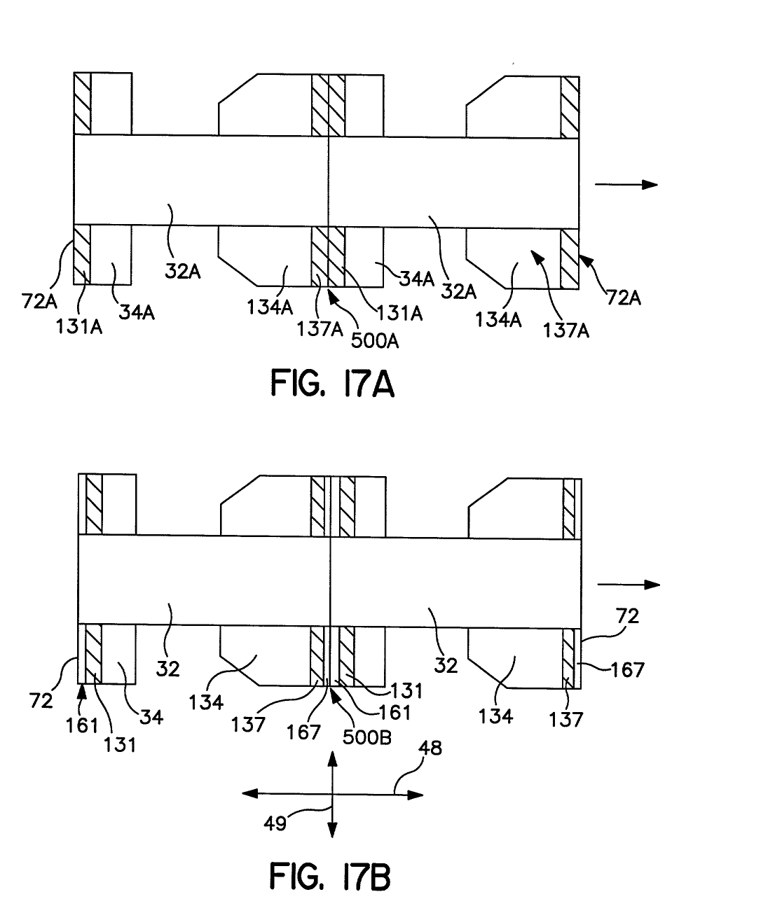

The term “machine direction assembly” refers to a process in which disposable absorbent products are manufactured in an orientation in which the products are connected end-to-end or waist-to-waist, in the longitudinal direction shown by arrow 48 in FIG. 17, a process utilizing a machine direction assembly entails products traveling through a converting machine parallel to the direction of arrow 48, as opposed to “cross-machine direction assembly” in which the products are connected side-to-side.

The term “spunbonded fibers” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Petersen, and U.S. Pat. No. 3,542,615 to Dobo et al. Spunbond fibers are quenched and generally not tacky on the surface when they enter the draw unit, or when they are deposited onto a collecting surface. Spunbond fibers are generally continuous and may have average diameters larger than 7 microns, often between about 10 and 30 microns.

The term “meltblown fibers” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than 10 microns in diameter, and are generally self bonding when deposited onto a collecting surface. Meltblown fibers used in the invention are suitably substantially continuous.

The term “polymer” generally includes but is not limited to, homopolymers, copolymers, including block, graft, random and alternating copolymers, terpolymers, etc., and blends and modifications thereof. Furthermore, unless otherwise specifically limited, the term “polymer” shall include all possible geometrical configurations of the material. These configurations include, but are not limited to isotactic, syndiotactic and atactic symmetries.

The term “substantially continuous filaments or fibers” refers to filaments or fibers prepared by extrusion from a spinnerette, including without limitation spunbonded and meltblown fibers, which are not cut from their original length prior to being formed into a nonwoven web or fabric. Substantially continuous filaments or fibers may have lengths ranging from greater than about 15 cm to more than one meter; and up to the length of the nonwoven web or fabric being formed. The definition of “substantially continuous filaments or fibers” includes those which are not cut prior to being formed into a nonwoven web or fabric, but which are later cut when the nonwoven web or fabric is cut.

The term “staple filaments or fibers” means filaments or fibers which are natural or which are cut from a manufactured filament prior to forming into a web, and which have a length ranging from about 0.1-15 cm, more commonly about 0.2-7 cm.

The term “fiber” or “fibrous” is meant to refer to a particulate material wherein the length to diameter ratio of such particulate material is greater than about 10. Conversely, a “nonfiber” or “nonfibrous” material is meant to refer to a particulate material wherein the length to diameter ratio of such particulate material is about 10 or less.

The term “thermoplastic” is meant to describe a material that softens when exposed to heat and which substantially returns to its original condition when cooled to room temperature.

The term “recover” or “retract” relates to a contraction of a stretched material upon termination of a biasing force following stretching of the material by application of the biasing force.

The term “garment” includes personal care garments, protective garments, and the like. The term “disposable garment” includes garments which are typically disposed of after 1-5 uses.

The term “personal care garment” includes diapers, training pants, swim wear, absorbent underpants, adult incontinence products, feminine hygiene products, and the like.

The term “protective garment” includes protective (i.e., medical and/or industrial) gowns, caps, gloves, drapes, face masks, and the like.

The term “in the vicinity of garment openings” refers to a zone or region of the garment within about two inches, suitably within about one inch, of a garment opening, such as a leg or waist opening. A zone or region is said to be “in the vicinity of a garment opening” if any portion of the zone or region is within two inches, suitably within one inch of the garment opening.

The term “aligned with a garment opening or edge” refers to a targeted elastic region (i.e., a high tension zone or TEM) that is parallel, or within plus or minus 30 degrees of parallel, to a garment edge defining a garment opening or to an edge of a garment.

The term “abutting a garment edge or opening” refers to a targeted elastic material, the cut edge of which is coincident with the cut edge of the garment.

The term “series” refers to a set including one or more elements.

### DETAILED DESCRIPTION OF THE PRESENTLY PREFERRED EMBODIMENTS

The principles of this invention can be applied to a wide variety of garments, including disposable garments, having a targeted elastic zone aligned with at least one garment opening or edge. Examples include diapers, training pants, certain feminine hygiene products, adult incontinence products, face masks, and other personal care or medical garments, and the like. For ease of explanation, the following description is in terms of a child training pant having a targeted elastic material, in this case a targeted elastic laminate, used for the side panels.

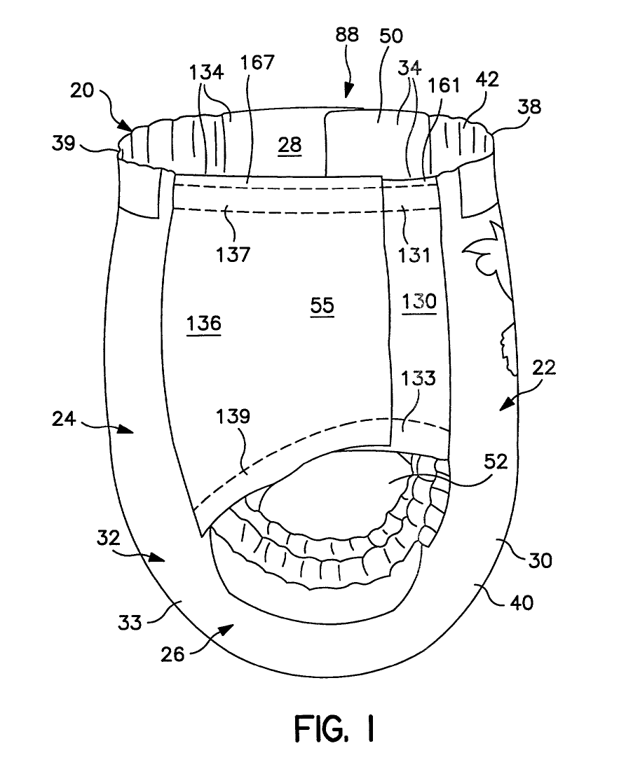

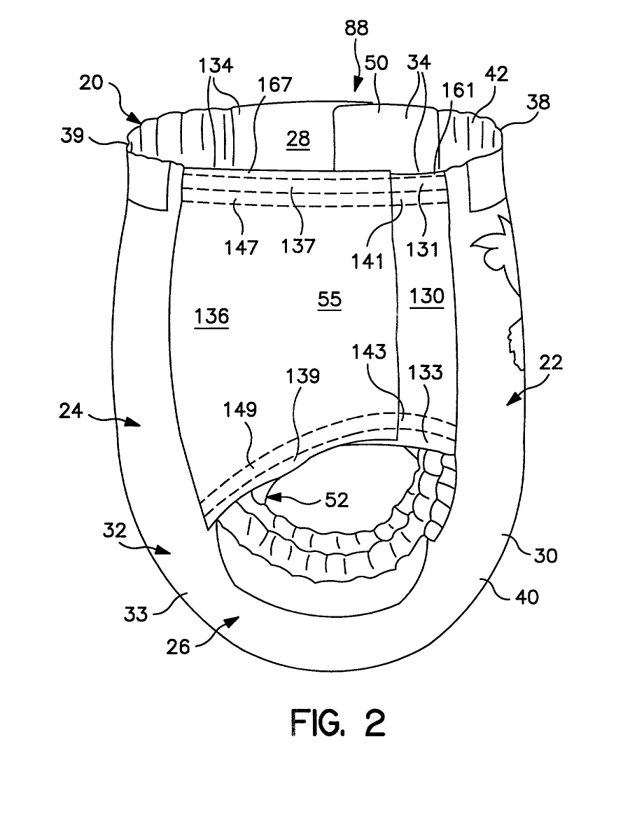

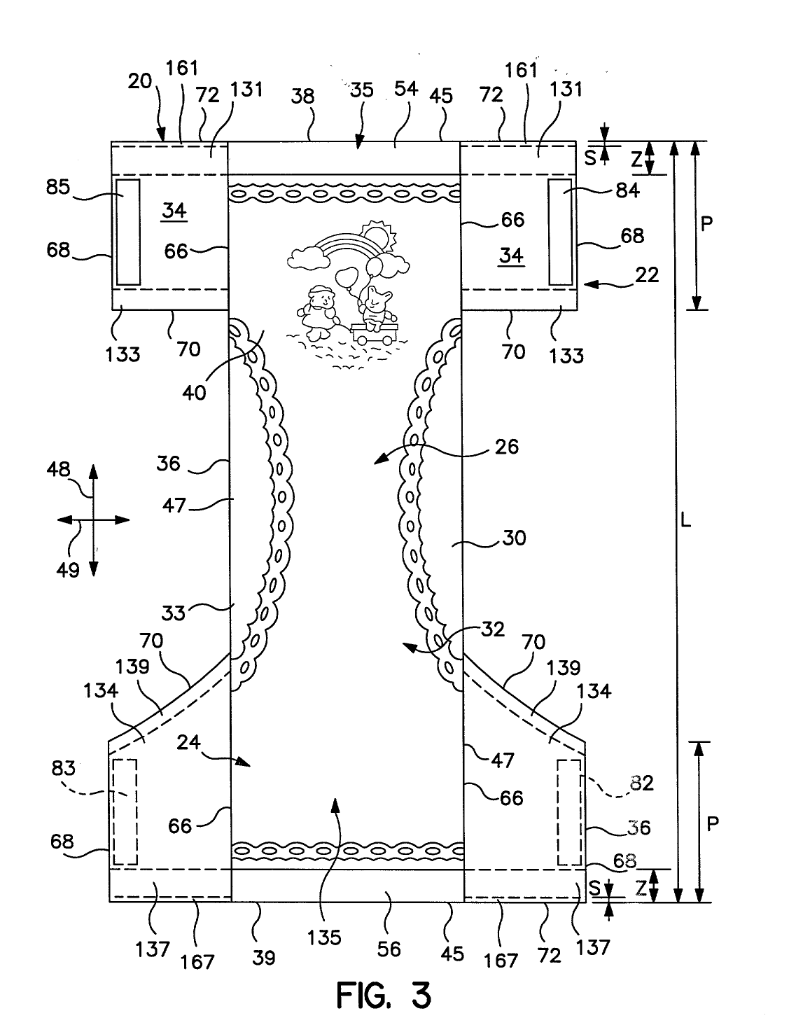

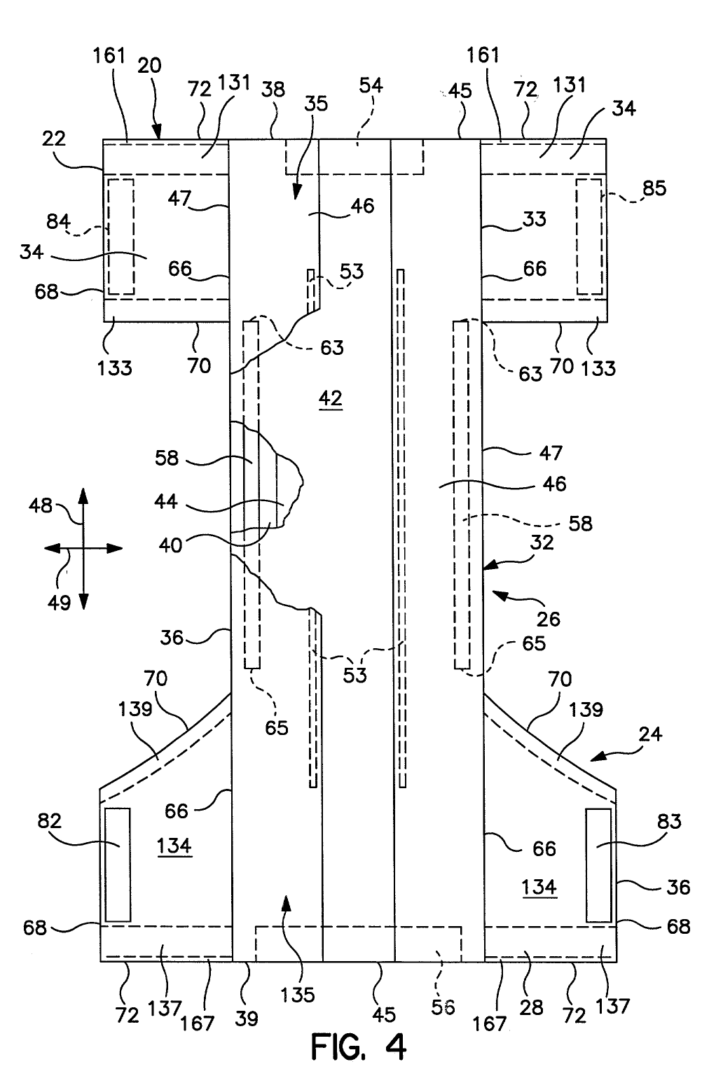

Referring to FIGS. 1 and 2, a disposable absorbent garment 20, such as a child training pant, includes an absorbent chassis 32 and a fastening system 88. The absorbent chassis 32 defines a front waist region 22, a back waist region 24, a crotch region 26 interconnecting the front and back waist regions, an inner surface 28 which is configured to contact the wearer, and an outer surface 30 opposite the inner surface which is configured to contact the wearer's clothing. With additional reference to FIGS. 3 and 4, the absorbent chassis 32 also defines a pair of transversely opposed side edges 36 and a pair of longitudinally opposed waist edges, which are designated front waist edge 38 and back waist edge 39. The front waist region 22 is contiguous with the front waist edge 38, and the back waist region 24 is contiguous with the back waist edge 39. The chassis 32 defines a waist opening 50 and two opposing leg openings 52.

The illustrated absorbent chassis 32 includes a somewhat rectangular absorbent composite structure 33, a pair of transversely opposed front side panels 34, and a pair of transversely opposed back side panels 134. The composite structure 33 and side panels 34 and 134 can be integrally formed or can include two or more separate elements, as shown in FIG. 1. The illustrated composite structure 33 includes an outer cover 40, a bodyside liner 42 (FIGS. 1 and 4) which is connected to the outer cover in a superposed relation, an absorbent assembly 44 (FIG. 4) which is located between the outer cover and the bodyside liner, and a pair of containment flaps 46 (FIG. 4). The somewhat rectangular composite structure 33 has opposite linear end edges 45 that form portions of the front and back waist edges 38 and 39, and opposite linear or curvilinear side edges 47 that form portions of the side edges 36 of the absorbent chassis 32 (FIGS. 3 and 4). For reference, arrows 48 and 49 depicting the orientation of the longitudinal axis and the transverse axis, respectively, of the training pant 20 are illustrated in FIGS. 3 and 4.

With the training pant 20 in the fastened position as illustrated in FIG. 1, the front and back waist regions 22 and 24 are joined together to define a three-dimensional pant configuration having a waist opening 50 and a pair of leg openings 52. The front waist region 22 includes the portion of the training pant 20 which, when worn, is positioned on the front of the wearer while the back waist region 24 includes the portion of the training paint which, when worn, is positioned on the back of the wearer. The crotch region 26 of the training pant 20 includes the portion of the training pant which, when worn, is positioned between the legs of the wearer and covers the lower torso of the wearer. The front and back side panels 34 and 134 include the portions of the training pant 20 which, when worn, are positioned on the hips of the wearer.

The front waist region 22 of the absorbent chassis 32 includes the transversely opposed front side panels 34 and a front center panel 35 (FIGS. 3 and 4) positioned between and interconnecting the side panels. The back waist region 24 of the absorbent chassis 32 includes the transversely opposed back side panels 134 and a back center panel 135 (FIGS. 3 and 4) positioned between and interconnecting the side panels. The waist edges 38 and 39 of the absorbent chassis 32 are configured to encircle the waist of the wearer when worn and provide the waist opening 50 which defines a waist perimeter dimension. Portions of the transversely opposed side edges 36 in the crotch region 26 generally define the leg openings 52.

The absorbent chassis 32 is configured to contain and/or absorb any body exudates discharged from the wearer. For example, the absorbent chassis 32 desirably although not necessarily includes the pair of containment flaps 46 which are configured to provide a barrier to the transverse flow of body exudates. A flap elastic member 53 (FIG. 4) is operatively joined with each containment flap 46 in any suitable manner as is well known in the art. The elasticized containment flaps 46 define an unattached edge which assumes an upright, generally perpendicular configuration in at least the crotch region 26 of the training pant 20 to form a seal against the wearer's body. The containment flaps 46 can be located along the transversely opposed side edges of the absorbent chassis 32, and can extend longitudinally along the entire length of the absorbent chassis or can only extend partially along the length of the absorbent chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pant 20 desirably includes a front waist elastic member 54, a rear waist elastic member 56, and leg elastic members 58, as are known to those skilled in the art (FIG. 4). The waist elastic members 54 and 56 can be operatively joined to the outer cover 40 and/or bodyside liner 42 along the opposite waist edges 38 and 39, and can extend over part or all of the waist edges. The leg elastic members 58 are desirably operatively joined to the outer cover 40 and/or bodyside liner 42 along the opposite side edges 36 and positioned in the crotch region 26 of the training pant 20. The leg elastic members 58 are desirably longitudinally aligned along each side edge 47 of the composite structure 33. Each leg elastic member 58 has a front terminal point 63 and a back terminal point 65, which points represent the longitudinal ends of the elastic gathering caused by the leg elastic members. The front terminal points 63 are desirably located adjacent the longitudinally innermost parts of the front side panels 34, and the back terminal points 65 are desirably located adjacent the longitudinally innermost parts of the back side panels 134.

The flap elastic members 53, the waist elastic members 54 and 56, and the leg elastic members 58 can be formed of any suitable elastic material, such as the targeted elastic material of the invention or separately manufactured and separately attached elastic materials. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat; such that elastic constrictive forces are imparted to the substrate. In one particular embodiment, for example, the leg elastic members 58 include a plurality of dry-spun coalesced multifilament spandex elastomeric threads sold under the trade name LYCRA® and available from E. I. Du Pont de Nemours and Company, Wilmington, Del., U.S.A., and other components of the garment, such as the side panels 55, include the targeted elastic material of the invention.

In the embodiment shown in FIG. 1, the front and back side panels 34 and 134 are fastened together by fastening system 88 to form collective side panels 55 (with each collective side panel 55 including a front side panel 34 and back side panel 134). In alternate embodiments, the collective side panels 55 can be single-piece side panels, or can include more than one piece permanently joined together. The transversely opposed front side panels 34 and transversely opposed back side panels 134 can be permanently bonded to the composite structure 33 of the absorbent chassis 32 in the respective front and back waist regions 22 and 24. More particularly, as shown best in FIGS. 3 and 4, the front side panels 34 can be permanently bonded to and extend transversely beyond the linear or curvilinear side edges 47 of the composite structure 33 in the front waist region 22 along attachment lines 66, and the back side panels 134 can be permanently bonded to and extend transversely beyond the linear or curvilinear side edges 47 of the composite structure 33 in the back waist region 24 along attachment lines 66. The side panels 34 and 134 can be attached using attachment means known to those skilled in the art such as adhesive, thermal or ultrasonic bonding. The side panels 34 and 134 can also be formed as a portion of a component of the composite structure 33, such as the outer cover or the bodyside liner. The fastening system 88 can include a plurality of fastener tabs 82, 83, 84 and 85, which can be known hook-and-loop fastener members, or other types of mechanical fasteners or adhesive fasteners. Alternatively, the front and back side panels 34, 134 can be permanently bonded together.

The illustrated side panels 34 and 134 each define a distal edge 68 that is spaced from the attachment line 66, a leg end edge 70 disposed toward the longitudinal center of the training pant 20, and a waist end edge 72 disposed toward a longitudinal end of the training pant. The leg end edge 70 and waist end edge 72 extend from the side edges 47 of the composite structure 33 to the distal edges 68. The leg end edges 70 of the side panels 34 and 134 form part of the side edges 36 of the absorbent chassis 32. In the back waist region 24, the leg end edges 70 are desirably although not necessarily angled relative to the transverse axis 49 to provide greater coverage toward the back of the pant as compared to the front of the pant. The waist end edges 72 are desirably parallel to the transverse axis 49. The waist end edges 72 of the front side panels 34 form part of the front waist edge 38 of the absorbent chassis 32, and the waist end edges 72 of the back side panels 134 form part of the back waist edge 39 of the absorbent chassis.

In particular embodiments for improved fit and appearance, the side panels 34 and 134 desirably have an average length dimension P measured parallel to the longitudinal axis 48 that is about 20% or greater, and particularly about 26% or greater, of the overall length dimension L of the absorbent garment, also measured parallel to the longitudinal axis 48 (FIG. 3). For example, in training pants having an overall length dimension of about 52 centimeters, the side panels 34 and 134 desirably have an average length dimension of about 10 centimeters or greater, such as about 14 centimeters. While each of the side panels 34 and 134 extend from the waist opening 50 to one of the leg openings 52, the back side panels 134 have a continually decreasing length dimension moving from the attachment line 66 to the distal edge as is best shown in FIGS. 3 and 4.

In accordance with particular embodiments of the invention, the front side panels 34 each include a targeted elastic material including a main body (low tension) zone 130, a narrow band-like high tension zone 131 aligned with waist opening 50, a narrow band-like high tension zone 133 in the vicinity of (and aligned with) the leg opening 52, and a narrow band-like spacer zone 161 in the vicinity (and aligned with) waist opening 50. The targeted elastic material abuts the waist end edge 72. Spacer zone 161 can be a second low tension zone or a no tension zone. The dotted lines indicate the boundaries between the low tension zone 130, high tension zones 131 and 133 and spacer zone 161, which boundaries may not be visible to an observer. From the standpoint of the observer, the TEM forming front side panels 34 may appear as a homogeneous, integrated material. Similarly, the rear side panels 134 each include a targeted elastic material including a main body (low tension) zone 136, a narrow, band-like high tension zone 137 aligned with waist opening 50, and a narrow, band-like high tension zone 139 in the vicinity of (and aligned with) the leg opening 52 and a narrow band-like spacer zone 167 in the vicinity (and aligned with) waist opening 50. Again, the targeted elastic material abuts the waist end edge 72. Spacer zone 167 can also be a second low tension zone or a no tension zone. Again, the dotted lines indicate invisible boundaries between the low tension zone 136, high tension zones 137 and 139 and spacer zone 167. The invention encompasses garments in which a high tension elastic zone is present and aligned with any one or more garment openings and/or edges. In an alternative embodiment, the front side panels 34 and back side panels 134 can further include a spacer zone aligned with each leg opening 52.

In particular embodiments for fit and appearance, each spacer zone (161 and 167) has a longitudinal length S measured parallel to the longitudinal axis 48 (FIG. 3). The length S can be in a range from about 0.2% to about 5% of the length L of the absorbent garment 20. The length S can suitably be in a range from about 0.4% to about 4% of the length L of the absorbent garment 20. Alternatively, the length S can be in a range from about 0.6% to about 2% of the length L of the absorbent garment 20. The length S for spacer zone 161 and the length S for spacer zone 167 may, but need not, be the same. Table 1 below shows how these percentages, in particular embodiments, correspond to a length S in mm for a training pant having an overall longitudinal length dimension L, defined between the front waist edge 38 and the back waist edge 39, of 52 cm, for example:

TABLE 1

| | |
|---|---|
| 0.2%-5% | 1 mm-26 mm |
| 0.4%-4% | 2 mm-21 mm |
| 0.6%-2% | 3 mm-10 mm |

In addition, the combined longitudinal length Z of each spacer zone (161 and 167) and its corresponding high tension zone (131 and 137) measured parallel to the longitudinal axis 48 (FIG. 3) can be less than about 15% of the length L of the absorbent garment 20. Each combined length Z can suitably be less than about 11% of the length L of the absorbent garment 20. Alternatively, each combined length Z can be less than about 8% of the length L of the absorbent garment 20. Table 2 below shows how these percentages, in particular embodiments, correspond to a length Z in mm for a training pant having an overall length dimension L of 52 cm:

TABLE 2

| | |
|---|---|
| 15% | 78 mm |
| 11% | 57 mm |
| 8% | 42 mm |

As shown in FIGS. 1, 3 and 4, the high tension zones 131 and 137 can be aligned end-to-end with waist elastics 54 and 56 on the front and back of chassis 32, to implement a performance similar to a continuous, or substantially continuous elastic band encircling the waist opening 50. Similarly, high tension zones 133 and 139 in the vicinity of leg openings 52 can be aligned with leg elastics 58, to implement a performance similar to a continuous, or substantially continuous elastic band encircling the leg openings. In the embodiments shown, waist elastic bands are aligned end-to-end with high tension zones on the TEM to create this function, with the use of TEM being limited to the front and back side panels. In other embodiments, and other garments, high tension zones of a TEM can encircle an entire garment opening, to give the performance of an elastic band without using one.

The high tension zones 131, 133, 137 and 139 exhibit greater elastic tension than the main portions 130 and 136 and the spacer zones 161 and 167 of side panels 34 and 134, without requiring the use of separately manufactured and attached elastic materials. The side panels 34 and 134 are manufactured from a targeted elastic material. Various embodiments of targeted elastic materials include the targeted elastic laminate materials shown in FIGS. 5-8. Referring to FIGS. 5A and 5B, TEL 100 (shown in sectional view, with the layers expanded apart from each other for clarity) includes a nonwoven layer 110 of elastomeric polymer filaments 108 made from a single elastic polymer or polymer blend, laminated to at least one, desirably two outer facing layers 120. TEL 100 includes a low tension central zone 102 (which can correspond to body region 136 in side panel 134 of FIG. 1), a first high tension end zone 104 (which can correspond to high tension zone 139 in FIG. 1), a second high tension end zone 106 (which can correspond to high tension zone 137 in FIG. 1), and a second low tension zone 125A (FIG. 5A) or a no tension zone 125B (FIG. 5B). The second low tension zone 125A or the no tension zone 125B can correspond to spacer zone 167 in FIG. 1. In the embodiment shown in FIG. 5B with no tension zone 125B, the elastomeric polymer filaments are absent from no tension zone 125B so that the no tension zone includes only the two outer facing layers 120. In the embodiment of FIGS. 5A and 5B, the polymer filaments 108 in the low tension zone 102 are spaced further apart and, thus define a lower basis weight per unit area of nonwoven layer 110. The polymer filaments 108 in the high tension zones 104 and 106 are spaced more closely together and, thus, define a higher basis weight per unit area of nonwoven layer 110. Except for the spacing between filaments (and the resulting variation in nonwoven web basis weight), the polymer filaments 108 can be identical in size and composition. The elastomeric nonwoven layer 110 can be stretched in the machine direction (i.e., a direction parallel to the longitudinal orientation of filaments 108) prior to bonding nonwoven layer 110 to the facing layers 120 using processes as described below. After the layers are bonded together, the laminate can be relaxed (allowing retraction) and extended again as needed. The TEL 100 can be used to manufacture side panels 34 and 134 as shown, or can be used in other portions of the garment chassis 32 so that the targeted elastic material abuts at least a portion of the waist opening 50.

In an alternative embodiment of the invention, zone 130 and/or 136 can be high tension zones, instead of low tension zones, and zones 137 and/or 131 can be low tension zones instead of high tension zones. Spacer zones 161 and 167 can be no tension zones, or if they are low tension zones, can have lower tension relative to low tension zones 137 and 131.

FIG. 2 illustrates an alternative embodiment of the garment of FIG. 1. Most of the elements in FIG. 2 are the same as in FIG. 1. As with FIG. 1, a TEL material can be used to form side panels 34 and 134. However, in FIG. 2, multiple high tension regions are shown in the vicinity of the waist and leg openings 50 and 52. In the front side panels 34, a first high tension zone 131 and a second high tension zone 141 and a spacer zone 161 are aligned with waist opening 50. A third high tension zone 133 and a fourth high tension zone 143 are aligned in the vicinity of leg opening 52. In the back side panels 134, a first high tension zone 137 and a second high tension zone 147 and a spacer zone 167 are aligned with waist opening 50. A third high tension zone 139 and a fourth high tension zone 149 are aligned in the vicinity of leg opening 52. The multiple high tension zones can have different levels of elastic tension, selected and tailored to optimize wearer comfort.

FIGS. 6-8 illustrate alternative embodiments of TEL materials which can be used to make the garment of FIG. 1 or FIG. 2. In FIG. 6, multiple high tension zones are present. Polymer filaments 108 in low tension zones 102 and 125A have relatively small diameters, and relatively large spacings between them. Polymer filaments 109 in outer high tension zones 104 and 106 have larger diameters than filaments 108, thus defining a higher nonwoven basis weight in zones 104 and 106. Polymer filaments 107 in inner high tension zones 114 and 116 have similar diameters but less inter-filament spacing than polymer filaments 108, again defining a higher nonwoven basis weight in zones 114 and 116 than in low tension zones 102 and 125A.

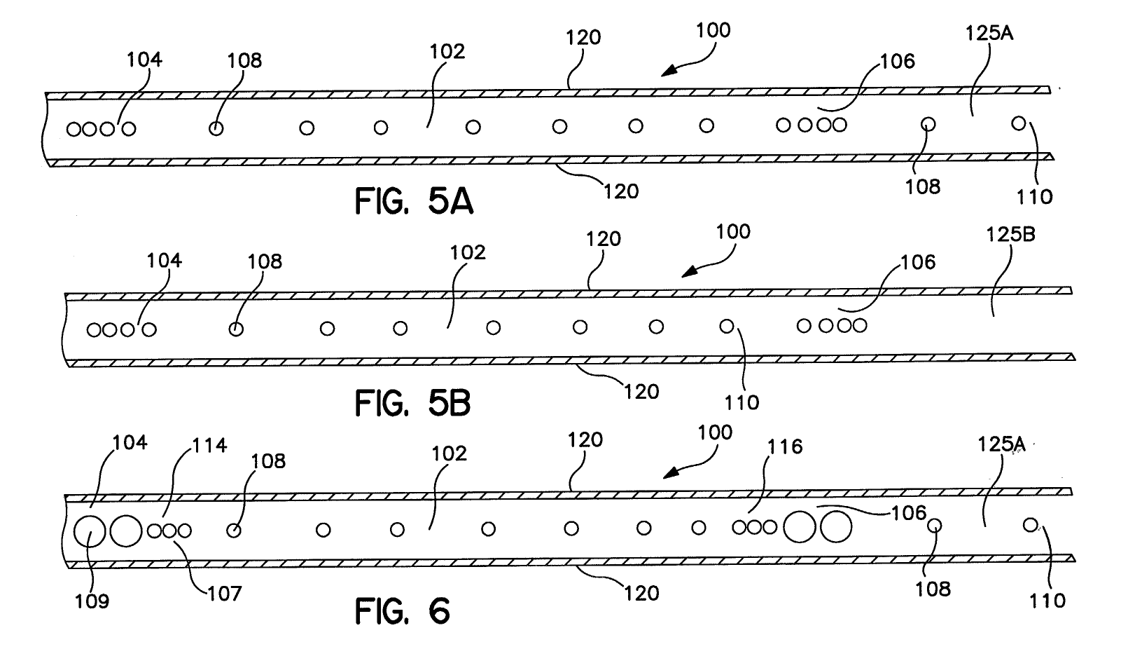

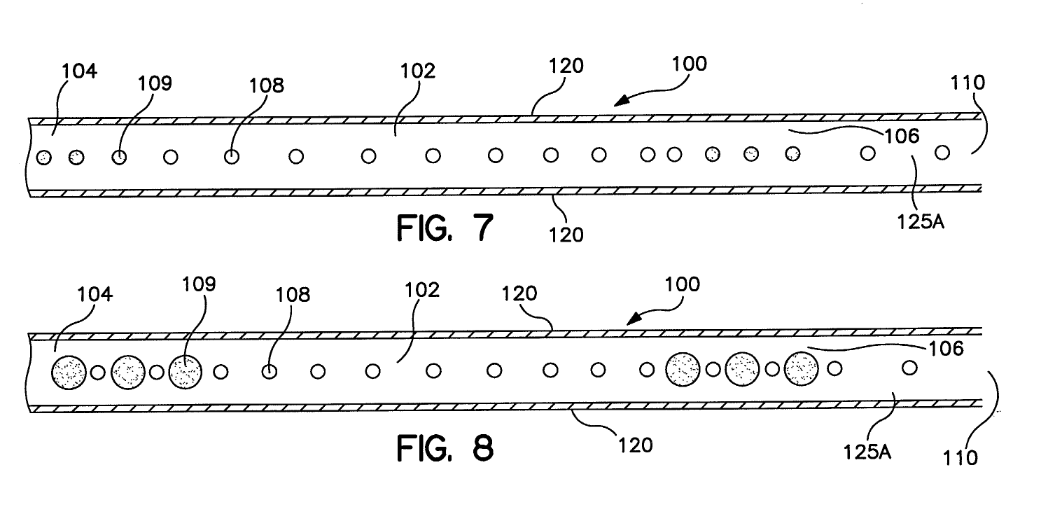

In the TEL of FIG. 7, the low and high tension zones 102, 125A, 104 and 106 are accomplished by forming the nonwoven layer 110 with two different elastic polymers or polymer blends, each one having a different elastic tension when stretched. The filaments 108 in low tension zones 102 and 125A are formed from a first elastic polymer or polymer blend having lower elastic tension. The filaments 109 in high tension zones 104 and 106 are formed from a second elastic polymer or polymer blend having higher elastic tension. Because different elastic polymers or polymer blends are used, the nonwoven layer 110 can have the same or different basis weights, the same or different filament sizes, and the same or different filament spacings in the low and high tension zones 102, 125A, 104 and 106.

The laminates of FIGS. 5-6 can each be produced by extruding the filaments 107, 108 and 109 of nonwoven layer 110 from a single die, having die plate openings sized and spaced to correspond to the desired filament sizes and spacing, or from different dies. The laminate of FIG. 7 can be produced by extruding filaments from either the same die fed by two or more polymer extruders, or from different dies for each polymer. Some of the processes described below illustrate how this is accomplished. In the laminate of FIG. 8, the nonwoven layer 110 can be formed by extruding two narrower bands of higher tension filaments 109 over a single wider band of lower tension filaments 108, using different dies and extruders. The result, shown in FIG. 8, is that low tension zones 102 and 125A contain only low tension filaments formed of a first elastic polymer or polymer blend. High tension zones 104 and 106 contain both high tension filaments 109 formed of a second elastic polymer or polymer blend, and low tension filaments 108.

In TEL 100, low tension zones 102 and 125A can have a first elastic tension, measured at 50% elongation of the filaments, and high tension zones 104 and 106 can have second and third elastic tensions higher than the first tension, measured at the same elongation. At 50% elongation of the TEL 100 (in the machine direction, parallel to filament orientation), high tension zones 104 and 106 can have an elastic tension at least 10% greater, suitably at least 50% greater, desirably 100-800% greater, alternatively about 125-500% greater, or as another alternative 150-300% greater than the low tension zone 102 or 125A. Elastic tension can be measured, for instance, using an MTS Sintech Model 1/s, sold by MTS in Research Triangle Park, N.C., with a crosshead speed set to 500 mm/mm. Samples having a 3-inch width and 6-inch length can be used, with 3 inches of the length clamped inside the jaws (leaving 3 inches of length for testing). The tension of each high and low tension region can be measured after the portion of the TEL laminate being tested is held in the extended condition (in the machine direction of the TEL) for 60 seconds.

In the TEL embodiments where the low and high tension zones are formed from nonwoven web sections having different basis weights (FIGS. 5-6), the nonwoven basis weights in the high tension zones 104 and 106 can be at least 10% greater, suitably at least 50% greater, or 100-800% greater, suitably 125-500% greater, or as another alternative 200-400% greater than the nonwoven basis weight in the low tension zones 102 and 125A. For instance, the nonwoven in the low tension zone can have a basis weight of about 2-14 grams per square meter (gsm), desirably about 4-12 gsm. In the high tension zones 104 and 106, the nonwoven basis weight can be about 10-32 gsm, desirably about 12-30 gsm. If the higher and lower basis weights are achieved using spinning holes of different frequency in the die, resulting in a higher areal density of filaments in the high tension regions and lower areal density of filaments in the low tension region, then the higher areal density can be at least 10% greater, suitably at least 50% greater, or 100-800% greater, suitably 125-500% greater, or as another alternative 200-400% greater than the lower areal density. The filament density in each zone can range from about 4-40 filaments per square inch (fsi), suitably about 12-30 fsi, measured perpendicular to the length of the filaments.

If the higher and lower basis weights are achieved using filaments of higher and lower diameters, as in FIG. 6, the higher diameter filaments 109 can have diameters at least 5% higher, suitably at least 20% higher, or 40-300% higher, alternatively 50-125% higher, or as another alternative 75-100% higher than the lower diameter filaments 108. The filament diameters in each zone can range from about 0.010-0.040 inch, suitably about 0.020-0.032 inch.

If the higher and lower tension zones are formed using nonwoven filaments 107, 108 and 109 of different elastic polymer composition, as shown in FIG. 7, then the different elastic polymers or polymer blends should be selected to give the desired higher elastic tension in the high tension zones 104 and 106 and the desired lower elastic tension in the low tension zones 102 and 125A. The nonwoven basis weights in the different zones can be the same or different, and can be adjusted, along with the polymer compositions, to achieve the desired elastic tensions. When a polymer blend is used, the blend itself should exhibit the desired elastic tension, regardless of the properties of the individual components.

Materials suitable for use in preparing elastomeric filaments 108 and 109 in the low and high tension zones 102, 125A, 104 and 106, include diblock, triblock, tetrablock or other multi-block elastomeric copolymers such as olefinic copolymers, including styrene-isoprene-styrene, styrene-butadiene-styrene, styrene ethylene/butylene-styrene, or styrene-ethylene/propylene-styrene, which can be obtained from the Shell Chemical Company, under the trade designation KRATON® elastomeric resin; polyurethanes, including those available from B. F. Goodrich Co., under the trade name ESTANE®; polyamides, including polyether block amides available from Ato Chemical Company, under the trade name PEBAX® polyether block amide; polyesters, such as those available from E. I. Du Pont de Nemours Co., under the trade name HYTREL® polyester; and single-site or metallocene-catalyzed polyolefins having density less than about 0.89 grams/cc, available from Dow Chemical Co. under the trade name AFFINITY®.

Alternatively, pre-made elastic materials such as elastic films or elastomeric threads (e.g., sold under the trade name LYCRA® and available from E. I. Du Pont de Nemours and Company, Wilmington, Del., U.S.A.) can be used to form elastomeric filaments 108 and 109.

A number of block copolymers can be used to prepare thermoplastic elastomeric filaments 108, 109 useful in this invention. Such block copolymers generally include an elastomeric midblock portion B and a thermoplastic endblock portion A, and are described in U.S. patent application Ser. No. 09/855,195, filed May 14, 2001, by Raymond J. May et al., which is incorporated herein by reference.

Other suitable elastomeric polymers can also be used to make thermoplastic elastomeric filaments 108, 109, and are also described in U.S. patent application Ser. No. 09/855,195, filed May 14, 2001 by Raymond J. May et al., which is incorporated herein by reference.

Elastic filaments 108 and 109 can also contain blends of elastic and inelastic polymers, or of two or more elastic polymers, provided that the blend exhibits elastic properties. The filaments can be substantially continuous or staple in length, but are desirably substantially continuous. Substantially continuous filaments have better elastic recovery than staple length filaments. Elastic filaments 107, 108 and 109 can be circular but can also have other cross-sectional geometries such as elliptical, rectangular, triangular or multi-lobal. In one embodiment, one or more of the filaments can be in the form of elongated, rectangular film strips produced from a film extrusion die having a plurality of slotted openings.

The facing layer or layers 120 can each include a nonwoven web, for example a spunbonded web or a meltblown web, a woven web, or a film. Facing materials can be formed using conventional processes, including the spunbond and meltblowing processes described in the “DEFINITIONS.” For example, facing materials 120 can include a spunbonded web having a basis weight of about 0.1-4.0 osy, suitably 0.2-2.0 osy, desirably about 0.4-0.6 osy. Each of the facing materials 120 can include the same or similar materials or different materials. In addition, either one or both of the facing layers 120 can be liquid-impermeable.

The facing materials 120 can be bonded to a nonwoven layer 110 (including the low and high tension zones thereof) using an adhesive, for example an elastomeric adhesive such as Findley H2525A, H2525 or H2096. Other bonding means well known to those having ordinary skill in the art can also be used to bond the facing materials 120 to filaments 108 and 109 of nonwoven layer 110, including thermal bonding, ultrasonic bonding, mechanical stitching and the like. Many of the same techniques can be used to bond the spacer zones 125A or 125B.

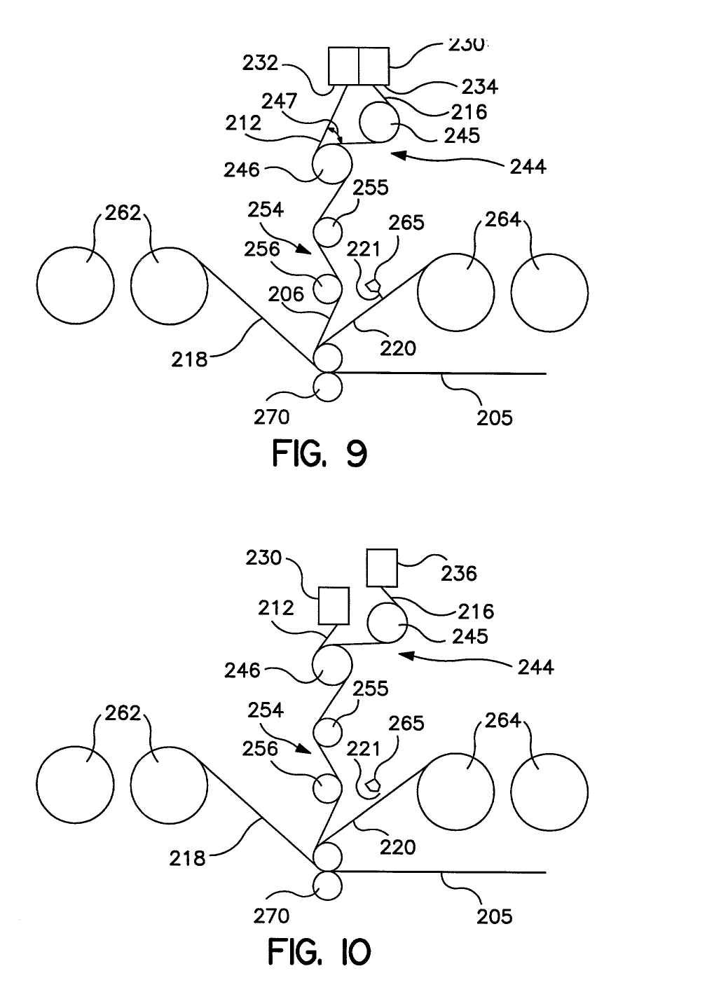

FIGS. 9-12 and 16 illustrate representative processes for making TEL materials. FIGS. 9 and 10 each illustrate a continuous vertical filament stretch-bond laminate (VF SBL) method. Referring to FIG. 9, an extruder (not shown) supplies molten elastomeric material to a first die 230. First die 230 includes different regions of spinning holes tailored to provide the nonwoven fabric 206 with higher and lower zones of elastic tension, having higher and lower basis weights or different polymer compositions as explained with respect to FIGS. 5-8.

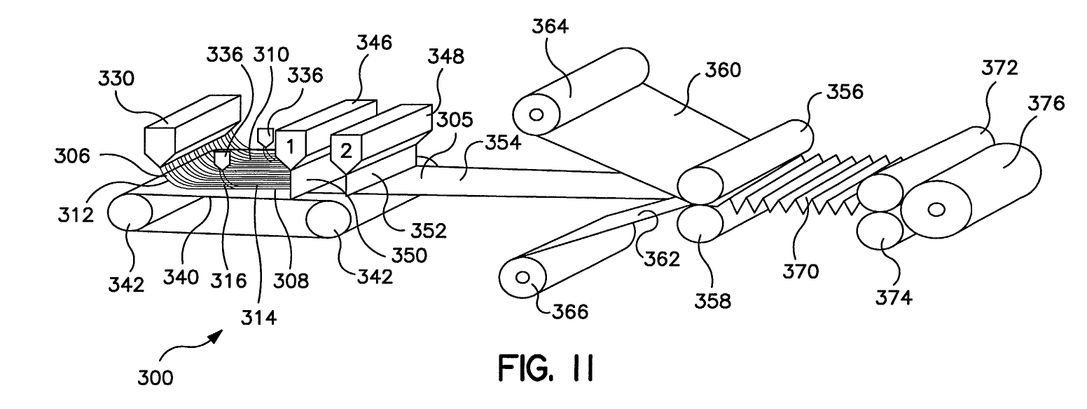

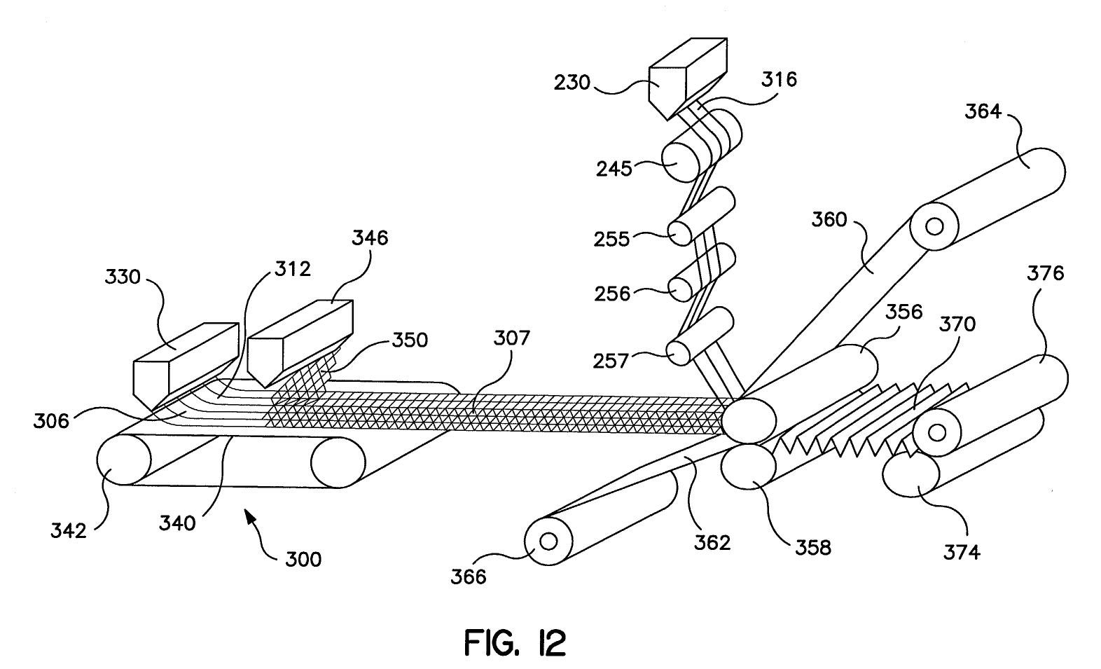

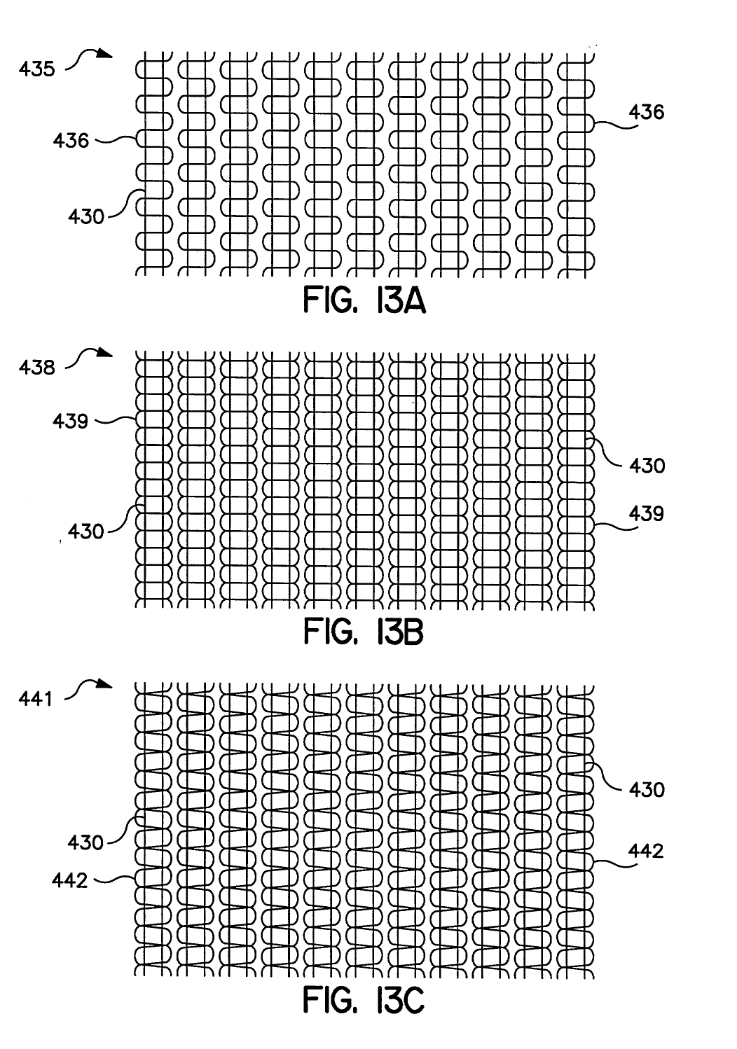

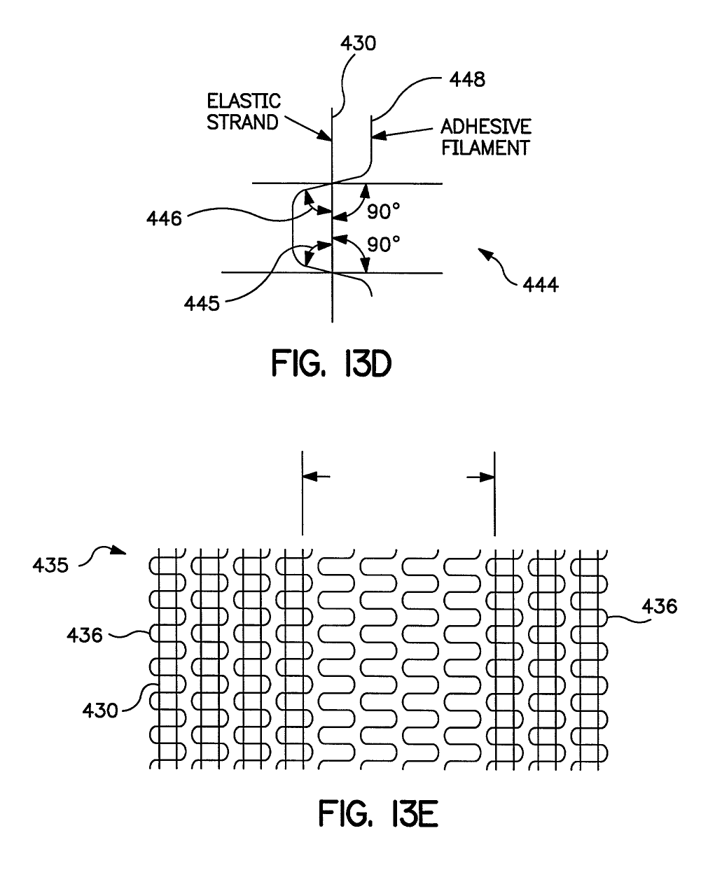

Referring to FIG. 9, molten elastomeric material is extruded from first spin plate region 232 through spinning holes as a plurality of elastomeric first filaments 212. Similarly, a plurality of elastomeric second filaments 216 are extruded from second spin plate region 234 through spinning holes of different average diameter, different frequency, and/or different polymer composition. The resulting nonwoven web 206 has a higher elastic tension in the zone defined by second filaments 216, than in the zone defined by first filaments 212. Second filaments 216 are positioned such that the high tension zone is divided into two or more zones, allowing for the intervening low or no tension zone. Filaments 212 can be excluded to form a no tension zone as shown in FIG. 13E. An example of the resulting laminate is shown in FIG. 5B (after the no tension zone has been cut in half to provide a single spacer zone on a garment between the high tension zone and the waist end edge of the side panel, as explained below). After extruding, first and second filaments 212 and 216 are quenched and solidified.

In one embodiment, first and second filaments 212 and 216 are quenched and solidified by passing them over a first series of chill rolls 244. For instance, first filaments 212 can be contacted with chill roll 246. Second filaments 216, having a higher aggregate basis weight, can be passed over two chill rolls 245 and 246. Any number of chill rolls can be used. Suitably, chill rolls 245 and 246 have a temperature of about 40° F. to about 80° F.

The die of each extruder can be positioned with respect to the first roller so that the continuous filaments meet this first roller at a predetermined angle 247. This strand extrusion geometry is particularly advantageous for depositing a melt extrudate onto a rotating roll or drum. An angled, or canted, orientation provides an opportunity for the filaments to emerge from the die at a right angle to the roll tangent point resulting in improved spinning, more efficient energy transfer, and generally longer die life. This improved configuration allows the filaments to emerge at an angle from the die and follow a relatively straight path to contact the tangent point on the roll surface. The angle 247 between the die exit of the extruder and the vertical axis (or the horizontal axis of the first roller, depending on which angle is measured) can be as little as a few degrees or as much as 90°. For example, a 90° extrudate exit to roller angle could be achieved by positioning the extruder directly above the downstream edge of the first roller and having a side exit die tip on the extruder. Moreover, angles such as about 20°, about 35°, or about 45° away from vertical can be utilized. It has been found that, when utilizing a 12-filament/inch spinplate hole density, an approximately 45° angle (shown in FIG. 9) allows the system to operate effectively. The optimum angle, however, will vary as a function of extrudate exit velocity, roller speed, vertical distance from the die to the roller, and horizontal distance from the die centerline to the top dead center of the roller. Optimal performance can be achieved by employing various geometries to result in improved spinning efficiency and reduced filament breakage. In many cases, this results in potentially increased roll wrap resulting in more efficient energy transfer and longer die life due to reduced drag and shear of the extrudate as it leaves the capillaries of the extruder die and proceeds to the chilled roll.

After first and second filaments 212 and 216 are quenched and solidified, they are stretched or elongated. In one desired embodiment, first and second filaments 212 and 216 are stretched using a first series of stretch rolls 254. First series of stretch rolls 254 can include one or more individual stretch rolls 255, desirably at least two stretch rolls 255 and 256, as shown in FIG. 9. Stretch rolls 255 and 256 rotate at a speed greater than a speed at which chill rolls 245 and 246 rotate, thereby stretching the nonwoven fabric 206, including the zones of first and second filaments 212 and 216.

In one embodiment, each successive roll rotates at a speed greater than the speed of the previous roll. For example, referring to FIG. 9, chill roll 245 rotates at a speed “x”; chill roll 246 rotates at a speed greater than “x”, for example about “1.1x”; stretch roll 255 rotates at a still greater speed, for example about “1.15x”; second stretch roll 256 rotates at a still greater speed, for example about “1.25x” to about “2x”; and a third stretch roll (not shown) rotates at a still greater speed, for example about “2x” to about “7x.” As a result, first and second filaments 212 and 216 can be stretched by about 100% to about 800% of an initial length, suitably by about 200% to about 700% of an initial length.

After first and second filaments 212 and 216 are stretched, elastic nonwoven web 206 is laminated to a first facing material 218 and (alternatively) a second facing material 220. First facing material 218 is unwound from one of the rollers 262 and laminated to a first side of nonwoven web 206. Second facing material 220 is unwound from one of the rollers 264 and laminated to a second side of nonwoven web 206. As shown in FIG. 9, before second facing material 220 is laminated to a second side of elastic nonwoven web 206, at least a portion of second facing material 220 can be coated or sprayed with an elastomeric adhesive 221, such as Findley H2525A, H2525 or H2096, via an adhesive sprayer 265. The laminate material is then passed through nip rolls 270 (desirably smooth calender rolls) and is relaxed and/or retracted to produce a TEL 205. Other means for bonding the laminate material known to those having ordinary skill in the art can be used in place of nip roll 270.

FIG. 10 illustrates a VF SBL process similar to that of FIG. 9. In FIG. 10, instead of using a single spinnerette 230 having adjacent die regions for the high and low tension filament zones, two spinnerettes 230 and 236 are employed. First spinnerette 230 extrudes the first filaments 212. Second spinnerette 236 extrudes the second filaments 216. Again, the first and second spinnerettes differ as to the aggregate basis weights and/or polymer compositions of the elastomeric filaments produced. The second spinnerette 236 can have die openings of a) higher frequency and/or b) higher diameter, than the die openings of the first spinnerette 230. Except for the use of two spinnerettes instead of one “hybrid” spinnerette, the processes of FIGS. 9 and 10 are similar. In either case, the first filaments 212 and second filaments 216 ultimately converge to form a single elastic nonwoven web 206 having zones of higher and lower elastic tensions. The filaments 212 and 216 can converge in a side-by-side fashion as shown in FIGS. 5-7, for instance, to produce zones of higher and lower tension. Alternatively, the bands of filaments 212 and 216 can have different widths such that a narrower layer or band of second filaments 216 is superimposed directly over a wider layer band of filaments 212, so that the higher tension zone occurs where the two layers coexist as exemplified in FIG. 8. In either process, the first filaments 212 and second filaments 216 can converge as shown, at the chill roll 246. Second filaments 216 can be positioned such that the high tension zone is divided into two or more zones, allowing for the intervening low or no tension zone. Filaments 212 can be excluded to form a no tension zone as shown in FIG. 13E. The laminate is shown in FIG. 5B (after the no tension zone has been cut in half to provide a single spacer zone on a garment between the high tension zone and the waist end edge of the side panel, as explained below).

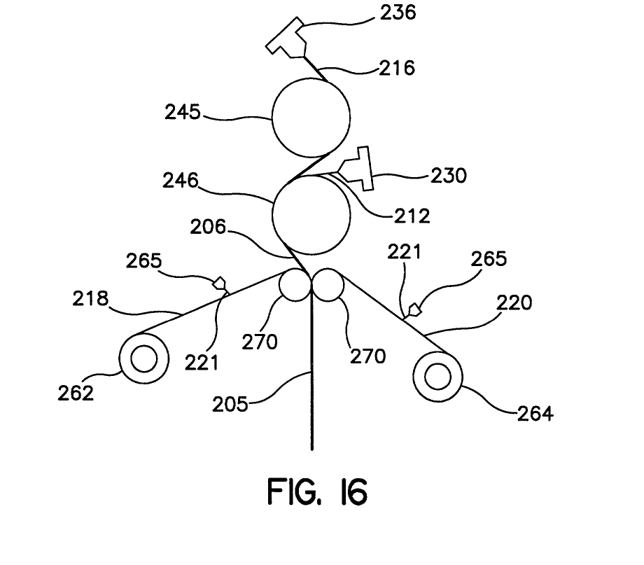

FIG. 16 illustrates a VF SBL process in which no stretch rolls 254 are used. Instead, first filaments 212 are extruded onto chill roll 246. Second filaments 216 are extruded onto chill roll 245. The first filaments 212 and second filaments 216 converge on chill roll 246 to form a single elastic nonwoven layer 206 having zones of higher and lower elastic tensions. Second filaments 216 can be positioned such that the high tension zone is divided into two or more zones, allowing for the intervening low or no tension zone. Filaments 212 can be excluded to form a no tension zone as shown in FIG. 13E. The laminate is shown in FIG. 5B (after the no tension zone has been cut in half to provide a single spacer zone on a garment between the high tension zone and the waist end edge of the side panel, as explained below). The first and second filaments 212, 216 are stretched between the chill rolls 245, 246 and the nip rolls 270. Except for the lack of stretch rolls 254, the processes of FIGS. 9 and 16 are similar. In either case, the elastic nonwoven layer 206 is laminated between a first facing layer 218 and a second facing layer 220 at the nip rolls 270. The resulting laminate is then relaxed and/or retracted to form TEL 205.

FIG. 11 illustrates a continuous horizontal filament stretch-bond laminate (CF SBL) process 300 for making TEL materials. A first extrusion apparatus 330 (which can be a spinnerette, as described above) is fed with an elastomeric polymer or polymer blend using one or more extruders (not shown). In various embodiments, the extrusion apparatus 330 can be configured to form a nonwoven layer 306 having zones of higher and lower elastic tension, as illustrated in FIGS. 5-7. In another embodiment, the extrusion apparatus 330 can be configured with die holes of uniform size and spacing, to yield a nonwoven layer 306 which has uniform elastic tension across its width. The nonwoven layer 306 contains filaments 312 which are substantially continuous in length. In this regard, the extrusion apparatus 330 can be a spinnerette. Suitably, apparatus 330 is a meltblowing spinnerette operating without the heated gas (e.g., air) stream which flows past the die tip in a conventional meltblowing process. Apparatus 330 extrudes filaments 312 directly onto a conveyor system, which can be a forming wire system 340 (i.e., a foraminous belt) moving clockwise about rollers 342. Filaments 312 can be cooled using vacuum suction applied through the forming wire system, and/or cooling fans (not shown). The vacuum can also assist in holding nonwoven layer 306 against the forming wire system.

In a desired embodiment, at least one, possibly two or more second extrusion apparatus 336 are positioned downstream of the first extrusion apparatus 330. The second extrusion apparatus create one or more higher tension zones in the nonwoven layer 306 by extruding filaments 316 of elastic material directly onto the nonwoven layer 306 in bands or zones which are narrower than the width of nonwoven layer 306. The second filaments 316 can be of the same elastic polymer construction as the first filaments 312. The extrusion of second filaments 316 over the first filaments 312 only in selected regions of layer 306, operates to create higher elastic tension zones 314 where the first and second filaments 312 and 316 coexist, and lower elastic tension zones 310 where the first filaments 312 exist alone. Second filaments 316 can be positioned such that the high tension zone is divided into two or more zones, allowing for the intervening low or no tension zone. Filaments 312 can be excluded to form a no tension zone as shown in FIG. 13E. The laminate is shown in FIG. 5B (after the no tension zone has been cut in half to provide a single spacer zone on a garment between the high tension zone and the waist end edge of the side panel). The first and second filaments 312 and 316 converge, and are combined in the forming conveyor 340 as it travels forward, to yield nonwoven layer 308 having at least one first zone 310 of lower elastic tension, and second, outer zones 314 of higher elastic tension.

As explained above, nonwoven layer 308 can be produced either a) directly from spinnerette 330, which is configured to yield zones of higher and lower elastic tension similar to FIGS. 5-7, or b) through the combined effect of spinnerette 330 as a uniform or nonuniform die, and secondary spinnerettes 336 which increase the elastic tension in localized regions of layer 308 by extruding secondary filaments 316 onto layer 306, similar to the web in FIG. 8. In either case, the nonwoven layer 308 (including filaments 312 and 316) can be incrementally stretched and, to an extent, maintained in alignment by moving the foraminous conveyor 340 in a clockwise machine direction, at a velocity which is slightly greater than the exit velocity of the filaments leaving the die.

To make the TEL 305, the elastic nonwoven layer 308 having higher and lower elastic tension zones is reinforced with one or more elastomeric meltblown layers made of the same or different elastic polymer material. Referring to FIG. 11, meltblowing extruders 346 and 348 are used to form meltblown layers 350 and 352 onto one side of layer 308, resulting in TEL 305. The meltblown layer or layers can act as structural facing layers in the laminate, and/or can act as tie layers if it is desired to add still more layers to the laminate.

In alternative embodiments, the targeted elastic material can be manufactured by joining together pre-existing, free-standing materials. The free-standing material can be an elastomer already laminated to a facing, or a woven elastic band.

Several patents describe various spray apparatuses and methods that can be utilized in supplying the meltblown layers (adhesives) to the outer facing(s) or, when desired, to the elastic strands themselves. For example, the following United States patents assigned to Illinois Tool Works, Inc. (“ITW”) are directed to various means of spraying or meltblowing fiberized hot melt adhesive onto a substrate: U.S. Pat. Nos. 5,882,573; 5,902,540; 5,904,298. These patents are incorporated herein in their entireties by reference thereto. The types of adhesive spray equipment disclosed in the aforementioned patents are generally efficient in applying the adhesive onto the nonwoven outer facings in the VFL process of this invention. In particular, ITW—brand Dynatec spray equipment, which is capable of applying about 3 gsm of adhesive at a run rate of about 1100 fpm, can be used in the melt-spray adhesive applications contemplated by the present inventive process.

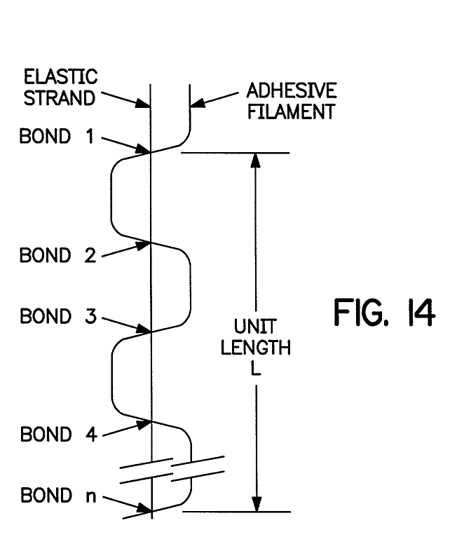

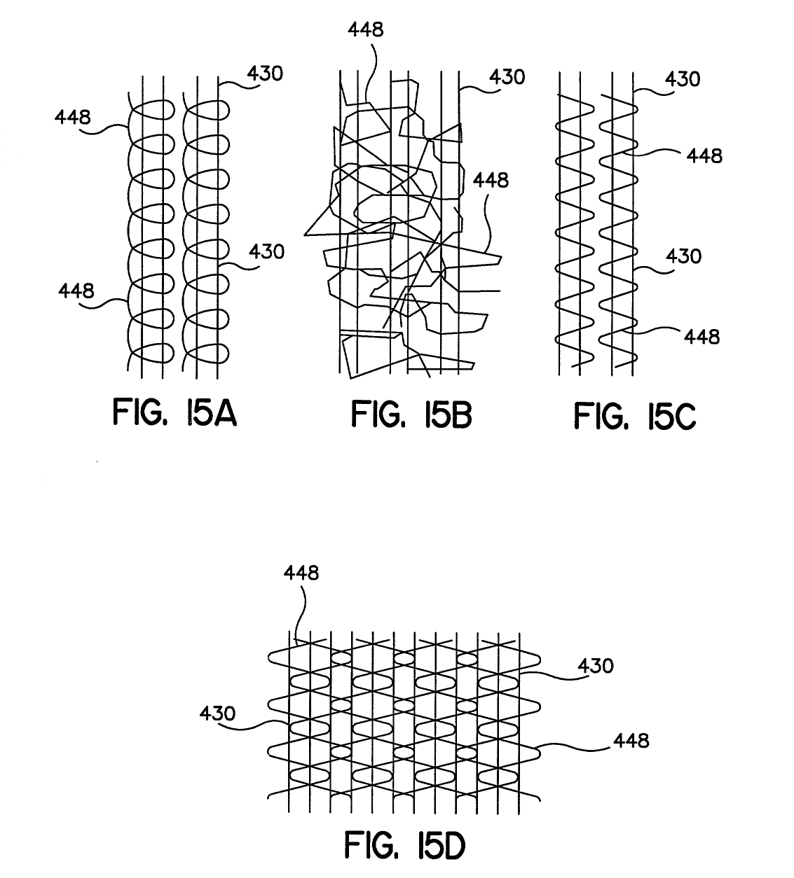

Representative adhesive patterns are illustrated in FIGS. 13A through 15E. Applying an adhesive in a cross-machine pattern such as the ones shown in FIGS. 15C and 15D can result in certain adherence advantages. For example, because the elastic strands are placed in the machine direction, having the adhesive pattern orient to a large degree in the cross-machine direction provides multiple adhesives to elastic crossings per unit length.

In addition, in many particular embodiments of the present invention, the adhesive component is applied to the surface of the nonwoven layer in discrete adhesive lines. The adhesive can be applied in various patterns so that the adhesive lines intersect the elastic filament lines to form various types of bonding networks which could include either adhesive-to-elastic bonds or adhesive-to-elastic bonds, adhesive-to-facing layer, and adhesive-to-adhesive bonds. These bonding networks can include a relatively large total number of adhesive-to-elastic and adhesive-to-adhesive bonds that provide the laminated article with increased strength, while utilizing minimal amounts of adhesive. Such enhancements are achieved by the use of adhesive sprayed onto the surface of the nonwoven in a predetermined and specific pattern. In most cases, a final product with less adhesive exhibits a reduction in undesirable stiffness, and is generally more flexible and soft than products having more adhesive.

Applying the adhesive in a pattern so that the adhesive lines are perpendicular or nearly perpendicular to the elastic components has been found particularly advantageous. A true 90° bond angle may not be possible in practice, but an average or mean bond angle that is as great as 50° or 60° will generally produce a suitable bond between the elastic strands and the facing material. A conceptual illustration of these types of bond angles is shown in FIGS. 13D and 14. The adhesive-to-elastic bonds are formed where the lines of adhesive 448 and elastic strands 430 join or intersect.

The continuous adhesive filaments-to-elastic strand intersections are also controlled to a predetermined number of intersections per unit of elastic strand length. By having such adhesive lines in a perpendicular orientation and optimizing the number of bonds per unit of elastic strand length, the final elastic strand laminate can be produced with a minimal amount of adhesive and elastomeric strand material to provide desirable product characteristics at a lower cost.

If the adhesive-to-elastic bonds are too few in number or are too weak, then the elastic tension properties of the laminate may be compromised and the tension applied to the elastic strands may break the adhesive joints. In various known processes, the common remedy for this condition is to increase the number of bonding sites by either increasing the meltspray air pressure, or by slowing the lamination speed. As the meltspray air pressure is increased, the resulting adhesive fiber size is reduced, creating weaker bonds. Increasing the amount of adhesive used per unit area to create larger adhesive filaments can strengthen these weaker bonds, which usually increases the cost of the laminate. Lowering the lamination speed decreases machine productivity, negatively impacting product cost. The present invention, in part, utilizes an effective bonding pattern where the number of bond sites per length elastic strand are prescribed and where the adhesive-to-elastic strand joints are generally perpendicular in orientation in order to provide maximum adhesive strength. This allows the laminate to be made at minimal cost by optimizing the adhesive and elastomer content to match the product needs.

As used herein, a “scrim” refers generally to a fabric or nonwoven web of material which can be elastic or inelastic, and having a machine direction (“MD”) oriented strand component along the path of product flow during manufacture and a cross-machine direction (“CD”) strand component across the width of the fabric.

FIG. 13A shows one exemplary scrim pattern useful in the present invention in which the adhesive has been applied to the elastic filaments with attenuation of the adhesive lines in the cross-machine direction. Scrim pattern 435 includes adhesive line 436 and elastic filaments 430. FIG. 13B illustrates another exemplary scrim pattern 438 having adhesive lines 439 applied to elastic strands 430. In this embodiment, it can be seen that the bond angle is very high, approaching 90° at the intersection between the adhesive and the elastic filaments. FIG. 13C illustrates still another scrim pattern 441 having adhesive lines 442 and continuous elastic strands 430.

As previously discussed, FIG. 13D illustrates the relatively high bond angle that can be employed in products produced according to the present invention. In particular, lay down angle 444 is shown as the angle formed by the adhesive line 448 and the elastic strand 430. Adhesive/elastic angle 446 and adhesive/elastic angle 445 are shown as being less than 90°.

FIG. 13E shows an example of a no tension zone located between the arrows. No elastic filaments are present in the no tension zone, however, adhesive continues to be used to laminate the two facing layers 120 (FIG. 5B; layers 120 are not shown in FIG. 13E). The type of adhesive pattern is not of particular relevance in the no tension zone.

FIG. 14 utilizes an exemplary bonding pattern to conceptually illustrate the measurement for determining the number of bonds per unit length on elastic strands or filaments. FIG. 15A shows another exemplary bonding pattern having the adhesive-to-adhesive bonding wherein a swirled type of configuration is employed. FIG. 15B illustrates a more randomized pattern wherein a large percentage of adhesive lines are in a perpendicular, or almost perpendicular, orientation to the elastic filaments. FIG. 15C is another exemplary embodiment of a bonding pattern having no adhesive-to-adhesive bonds, but numerous adhesive-to-elastic strand bonds. FIG. 15D illustrates another exemplary bonding pattern that has both adhesive-to-adhesive and adhesive-to-elastic strand bonds. The configuration shown in FIG. 15D is similar to the design of a chain-link fence.

Then, if it is desired to convert the TEL 305 into a stretch-bonded laminate, the TEL 305 can be stretched in a stretching stage 354 by pulling it between two nip rolls 356 and 358 which turn at a higher surface speed than the conveyor 340. At the same time, the facing layers 360 and 362 can be unwound from supply rollers 364 and 366, and laminated to the TEL 305 using the stretch roll assembly. To accomplish this dual purpose, the nip rolls 356 and 358 can be smooth or patterned calender rolls which use pressure to bond the materials 360, 305 and 362 together as well as stretch the TEL 305. Alternatively, both heat and pressure can be applied to bond the materials 360, 305 and 362 together. The resulting stretch-bonded laminate 370 can then be relaxed and/or retracted using nip rollers 372 and 374 that rotate at lower surface speed than calender rolls 358, and can be wound onto storage roll 376. The facing layers 360 and 362 can be any of the facing materials described above, and are desirably polyolefin-based spunbond webs.

FIG. 12 illustrates a hybrid 300 of a CF SBL process and a VF SBL process for making a stretch-bonded TEL 370. A first extrusion apparatus 330 is fed with an elastic polymer or polymer blend from one or more sources (not shown). Extrusion apparatus 330 can be any of the various devices described with respect to FIG. 11. Suitably, apparatus 330 is a meltblowing spinnerette operating without the heated gas (e.g., air) stream which flows past the die tip in conventional meltblowing processes. Apparatus 330 extrudes lower tension filaments 312 directly onto a conveyor system, which can be a forming wire system 340 (i.e., a foraminous belt) moving clockwise about rollers 342. Filaments 312 can be cooled using vacuum suction applied through the forming wire system, and/or cooling fans (not shown). The vacuum can also help hold the filaments against the forming wire system.

A meltblowing extruder 346 is used to add a reinforcing elastic meltblown layer 350 to the elastic filaments 312. Desirably, the meltblown layer 350 is made of the same elastic polymer as the low tension filaments 312. The resulting laminate 307 travels forward on the conveyor.

To make the higher tension region, a vertical filament die 230 extrudes higher tension (i.e., higher basis weight) elastic filaments 316 in a band which is narrower than the laminate 307 containing filaments 312. Filaments 316 pass around a chill roll 245, or a series of chill rolls, and a series of stretch rolls, for example two stretch rolls 255, 256, before being joined with laminate 307 between nip rolls 356 and 358, which are suitably smooth or patterned calender rolls. Simultaneously, facing layers 360 and 362 are unwound from supply rolls 364 and 366 and joined with the laminate between nip rolls 356 and 358 to make TEL 370. As TEL 370 is relaxed, it may assume the puckered configuration shown, due to retraction of high tension filaments 316 present in part of the laminate. TEL 370 can be flattened out between rolls 374 and 376, and wound onto roll 376.

The manufacture of training pants having side panels, with or without fastening components, can be accomplished in the manner described in U.S. patent application Ser. No. 09/855,484, filed May 15, 2001 by Joseph D. Coenen et al., which is incorporated herein by reference. FIG. 17A illustrates a machine direction disposable garment assembly in which the side panels 34, 134 are made of a targeted elastic material. A cutter cuts along cut line 500A to yield individual product assemblies with targeted elastic zones 137A and 131A with no spacer zone between the waist end edge 72A and the targeted elastic zones. Process variability may lead to targeted elastic zones 131A and 137A of variable sizes. FIG. 17B illustrates a machine direction disposable garment assembly in which the side panels are made of a targeted elastic material of the present invention. The cutter cuts along cut line 500 leaving spacer zone 161 and spacer zone 167 between the waist end edge 72 of the side panels and each targeted elastic zone 131 and 137. The presence of the spacer zones 161 and 167 allows the size of the targeted elastic zones to be manufactured consistently.

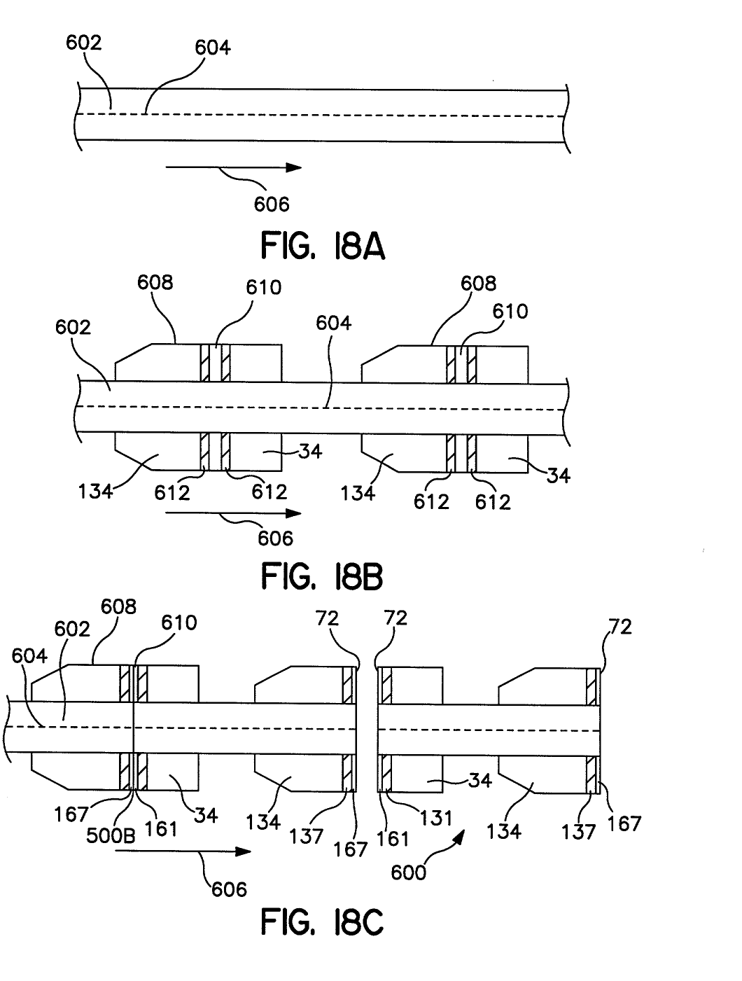

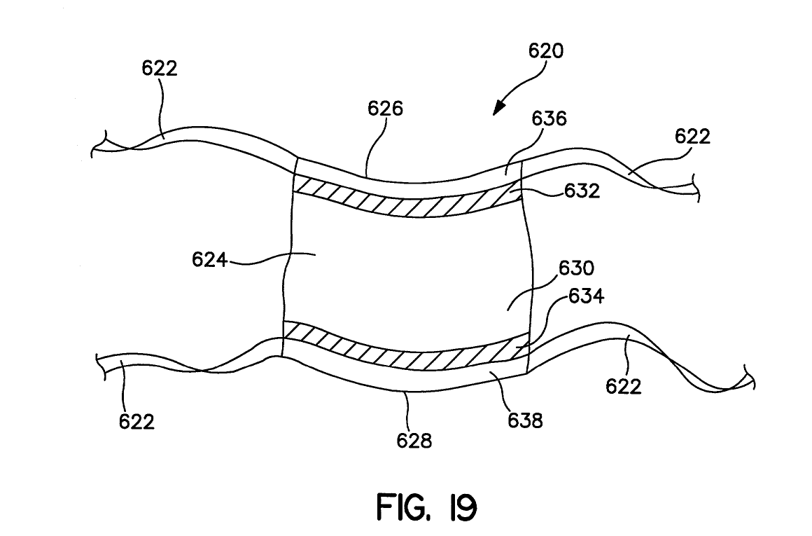

Referring to FIGS. 18A-18C, one embodiment of an assembly section for making a continuous stream of partially assembled, discrete training pants 600 is illustrated. The specific equipment and processes used in the assembly section can vary greatly depending on the specific type of garment being manufactured. The particular process and apparatus described in relation to FIGS. 18A-18C are specifically adapted to manufacture training pants 20 of the type illustrated in FIGS. 1-2, and are described in greater detail in U.S. patent application Ser. No. 09/855,484 mentioned above.

The various components of the training pant can be connected together by any means known to those skilled in the art such as, for example, adhesive, thermal and/or ultrasonic bonds. Desirably, most of the components are connected using ultrasonic bonding for improved manufacturing efficiency and reduced raw material costs. Certain garment manufacturing equipment which is readily known and understood in the art, including frames and mounting structures, ultrasonic and adhesive bonding devices, transport conveyors, transfer rolls, guide rolls, tension rolls, and the like, have not been shown in FIGS. 18A-18C. Suitable absorbent supply mechanisms, web unwinds, conveyor systems, registration systems, drives systems, control systems and the like, for use with the present process are disclosed in U.S. Pat. No. 5,766,389 issued Jun. 16, 1998 to Brandon et al., which is incorporated herein by reference.

As various components are added in the assembly section, a continuously moving product assemblage 602 is formed. The continuously moving product assemblage 602 defines a longitudinal center line 604 which can correspond to the machine center line. The product assemblage 602 will be cut downstream to form the partially assembled, discrete training pants 600.

The side panel material (e.g., the targeted elastic material of the present invention) can be cut into individual targeted elastic strips 608, and positioned partially on the continuously moving product assemblage 602 using an applicator device. One suitable applicator device is disclosed in U.S. Pat. Nos. 5,104,116 issued Apr. 14, 1992 and 5,224,405 issued Jul. 6, 1993 both to Pohjola, which are incorporated herein by reference. The targeted elastic strips can be side panel strips.

In addition, the targeted elastic strips can be any shape such as a square, rectangle, an irregular shape, or the like.

Referring to FIG. 18B, the targeted elastic strips 608 include a first zone 610, which corresponds to the combined size of spacer zones 161 and 167, as shown in FIGS. 1-4, and 18C, on the leading and training pants. The targeted elastic strips 608 also include two second zones 612, which correspond to high tension zones 131 and 137 as shown in FIGS. 1-4 and 18C. The first zone 610 is located between the second zones 612. In addition, the spacer zones 161 and 167 are located between the second zones 612 and the cut line 500B. The first zone 610 has lower tension than the second zones 612.

In the machine direction 606, the cutter is aligned with the first zone 610 and cuts the product assembly 602 within the first zone 610 along cut line 500B forming discrete garments with a front side panel 34 and a back side panel 134 on consecutive training pants 600, and spacer zones 161 and 167, thereby leaving the size of high tension zones 131 and 137 unaffected. The spacer zones 161 and 167 are between the waist end edge 72 and the high tension zones 131 and 137.

This method for making garments with spacer zones can be carried out in the machine direction as shown, or alternatively in the cross-machine direction, or in any other manner in which a first zone, corresponding to spacer zones, coincides with a cut line or edge (e.g., the waist end edge) of a garment.

The targeted elastic material of the present invention can be used in the side panels of training pants, or in any other appropriate portions of training pants, and can be manufactured separately from the processes for manufacturing the training pants. Thus, the targeted elastic material can be supplied as a pre-made good, such as on a roll, to a converting process for manufacturing the training pants. Alternatively, the targeted elastic material can be manufactured and converted into the training pants in the same machine.

The targeted elastic materials described above can be employed in a wide variety of personal care garments, and can be oriented and placed so that a high tension elastic region and a spacer zone are in the vicinity of at least one garment opening or edge. Suitable personal care garments having openings or edges include, for instance, diapers, training pants, swimwear, absorbent underpants, adult incontinence products, and certain feminine hygiene products. The targeted elastic materials can be used in similar fashion in protective garments including, for instance, medical gowns, gloves, caps, drapes, face masks, and the like, where it is desired to provide elastic properties aligned with one or more garment openings or edges without requiring a separately manufactured and attached elastic band.

Referring to FIG. 19, in another embodiment of the invention a garment, in this case a face mask 620, is shown. The face mask 620 includes ties 622, a face mask body 624, a top edge 626 and a bottom edge 628.

In accordance with this embodiment of the invention, the face mask 620 includes a targeted elastic material in the face mask body 624 including a low tension zone 630, a high tension zone 632 aligned with the top edge 626, a second high tension zone 634 aligned with the bottom edge 628, a spacer zone 636 aligned with top edge 626 and a second spacer zone 638 aligned with the bottom edge 628. The spacer zones 636 and 638 can be low tension zones or no tension zones. The spacer zone 636 is located between high tension zone 632 and the top edge 626. Similarly, the spacer zone 634 is located between high tension zone 634 and the bottom edge 628. The targeted elastic material abuts either the top edge 626, or the bottom edge 628, or both.

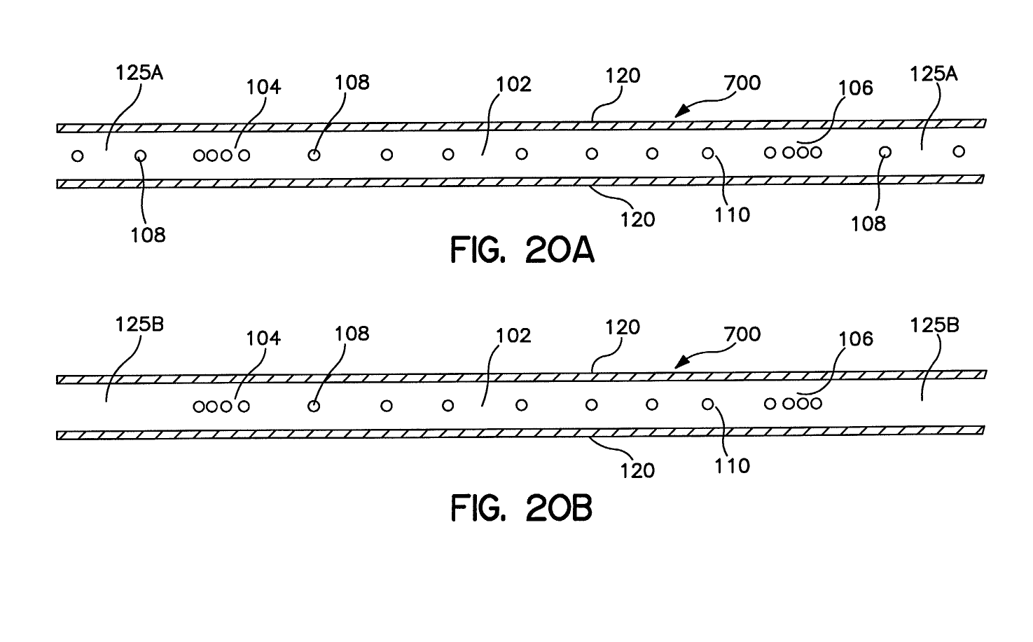

The high tension zones 632 and 634 exhibit greater elastic tension than the low tension zone 630 and the spacer zones, 636, 638, without requiring the use of separately manufactured and attached elastic materials. The face mask body 624 is manufactured from a targeted elastic material. Various embodiments of the targeted elastic material include the targeted elastic laminate materials shown in FIGS. 20A-20B. Referring to FIGS. 20A-20B, TEL 700 (shown in sectional view, with the layers expanded apart from each other for clarity) corresponds to previously described TEL 100 illustrated in FIGS. 5A and 5B. Reference numbers 102, 104, 106, 108, 110, 120, 125A and 125B correspond to the same numbers in FIGS. 5A and 5B, with the difference being that in FIG. 20A a second low tension zone 125A appears to the left of high tension zone 104 and in FIG. 20B a second no tension zone 125B appears to the left of high tension zone 104. Starting from the right of FIGS. 20A and 20B, low tension zone 125A or no tension zone 125B corresponds to spacer zone 636 (FIG. 19), high tension zone 106 corresponds to high tension zone 632, low tension zone 102 corresponds to low tension zone 630, high tension zone 104 corresponds to high tension zone 634 and second low tension zone 125A or no tension zone 125B corresponds to spacer zone 638.

Alternative embodiments of TEL materials can be used to make face mask body 624, for example, the TEL materials described and illustrated in FIGS. 6-8.

In an alternative embodiment of the invention according to FIG. 19, zone 630 can be a high tension zone, instead of a low tension zone, and zones 632 and 634 can be low tension zones instead of high tension zones. Spacer zones 636 and 638 can be no tension zones, or if they are low tension zones, can have lower tension relative to low tension zones 632 and 634.

It is contemplated that the garment of this embodiment is made of the targeted elastic material described herein with a spacer zone aligned with at least one edge. However, it is also contemplated that the elastic material in the garment may not be an integrated material as in the targeted elastic material described, but that the elastic material may be a conventional elastic material separately manufactured and attached to the body of the personal care garment so that the garment still includes a spacer zone aligned with and abutting an edge of the garment and an elastic zone, whether integrated or separately attached.

While the embodiments of the invention described herein are presently preferred, various modifications and improvements can be made without departing from the spirit and scope of the invention. The scope of the invention is indicated in the appended claims, and all changes that fall within the meaning and range of equivalents are intended to be embraced therein.

What is claimed:

1. A disposable absorbent garment having a longitudinal length defined between a front waist edge and a back waist edge, comprising:
a chassis including an absorbent composite structure and side panels extending from the absorbent composite structure;
a waist opening and a pair of leg openings defined by the chassis;
a waist end edge defined by each side panel; and
a targeted elastic material in the side panels, the targeted elastic material including at least one high tension zone aligned with the waist opening, at least one low tension zone, and a spacer zone between the at least one high tension zone and the waist end edge.

2. The disposable absorbent garment of claim 1, further comprising a high tension zone aligned with each leg opening.

3. The disposable absorbent garment of claim 1, wherein the targeted elastic material includes at least two high tension zones aligned with the waist opening.

4. The disposable absorbent garment of claim 3, further comprising a high tension zone aligned with each leg opening.

5. The disposable absorbent garment of claim 3, further comprising at least two high tension zones aligned with each leg opening.

6. The disposable absorbent garment of claim 1, wherein the spacer zone comprises a second low tension zone.

7. The disposable absorbent garment of claim 1, wherein the spacer zone comprises a no tension zone.

8. The disposable absorbent garment of claim 1, wherein the spacer zone has a longitudinal length in a range from about 0.2% to about 5% of the longitudinal length of the garment.

9. The disposable absorbent garment of claim 1, wherein the spacer zone and the at least one high tension zone have a combined longitudinal length less than about 15% of the length of the garment.

10. The disposable absorbent garment of claim 1, wherein the targeted elastic material comprises a targeted elastic laminate including the low and high tension zones and the spacer zone;
the low tension zone including a plurality of elastomeric first filaments, the low tension zone having a first basis weight;
the high tension zone including a plurality of elastomeric second filaments, the high tension zone having a second basis weight higher than the first basis weight;
the laminate further including a facing layer bonded to at least a first side of the low tension zone and a first side of the high tension zone.

11. The disposable absorbent garment of claim 10, wherein the facing layer is liquid-impermeable.

12. The disposable absorbent garment of claim 1, wherein the targeted elastic material comprises a targeted elastic laminate including the low and high tension zones and the spacer zone;
the low tension zone including a plurality of elastomeric first filaments, the first filaments including a first elastomeric polymer composition;
the high tension zone including a plurality of elastomeric second filaments, the second filaments including a second elastomeric polymer composition;
the laminate further including a facing material bonded to at least a first side of the low tension zone and a first side of the high tension zone.

13. The disposable absorbent garment of claim 1, wherein the targeted elastic material abuts the waist end edge.

14. A disposable garment, comprising:
a chassis defining a waist opening and two leg openings; and
a targeted elastic material in at least a portion of the chassis, the targeted elastic material including at least one high tension zone aligned with the waist opening, at least one low tension zone, and a spacer zone between the high tension zone and the waist opening.

15. The disposable garment of claim 14, wherein the spacer zone comprises a second low tension zone.

16. The disposable garment of claim 14, wherein the spacer zone comprises a no tension zone.

17. The disposable garment of claim 14, further comprising a high tension zone aligned with each leg opening.

18. The disposable garment of claim 14, wherein the targeted elastic material includes at least two high tension zones aligned with the waist opening.

19. The disposable garment of claim 18, further comprising a high tension zone aligned with each leg opening.

20. The disposable garment of claim 14, wherein the spacer zone is in the vicinity of the waist opening.

21. The disposable garment of claim 14, wherein the spacer zone has longitudinal length in a range from about 0.2% to about 5% of the longitudinal length of the garment.

22. The disposable garment of claim 14, wherein the spacer zone has a longitudinal length in a range from about 0.4% to about 4% of the longitudinal length of the garment.

23. The disposable garment of claim 14, wherein the spacer zone has a longitudinal length in a range from about 0.6% to about 2% of the longitudinal length of the garment.

24. The disposable garment of claim 14, wherein the spacer zone and the at least one high tension zone have a combined longitudinal length in the vicinity of the waist opening.

25. The disposable garment of claim 14, wherein the spacer zone and the at least one high tension zone have a combined longitudinal length less than about 15% of the length of the garment.

26. The disposable garment of claim 14, wherein the spacer zone and the at least one high tension zone have a combined longitudinal length less than about 11% of the length of the garment.

27. The disposable garment of claim 14, wherein the spacer zone and the at least one high tension zone have a combined longitudinal length less than about 8% of the length of the garment.

28. The disposable garment of claim 14, wherein the targeted elastic material comprises a targeted elastic laminate including the low and high tension zones and the spacer zone;
the low tension zone including a plurality of elastomeric first filaments, the low tension zone having a first basis weight;
the high tension zone including a plurality of elastomeric second filaments, the high tension zone having a second basis weight higher than the first basis weight;
the laminate further including a facing layer bonded to at least a first side of the low tension zone and a first side of the high tension zone.

29. The disposable garment of claim 14, wherein the targeted elastic material comprises a targeted elastic laminate including the low and high tension zones and the spacer zone;
the low tension zone including a plurality of elastomeric first filaments, the first filaments including a first elastomeric polymer composition;
the high tension zone including a plurality of elastomeric second filaments, the second filaments including a second elastomeric polymer composition;
the laminate further including a facing material bonded to at least a first side of the low tension zone and a first side of the high tension zone.

30. The disposable garment of claim 14, wherein the targeted elastic material comprises elastomeric filaments comprising elastic films.

31. The disposable garment of claim 14, wherein the targeted elastic material comprises elastomeric filaments comprising elastomeric threads.

32. The disposable garment of claim 14, comprising a diaper.

33. The disposable garment of claim 14, comprising a training pant.

34. The disposable garment of claim 14, comprising a feminine hygiene article.

35. The disposable garment of claim 14, comprising swim wear.

36. The disposable garment of claim 14, comprising an absorbent underpant.

37. The disposable garment of claim 14, wherein the targeted elastic material abuts at least a portion of the waist opening.

38. A disposable garment having a longitudinal length defined between a front waist edge and a back waist edge, comprising:
a chassis including side panels and defining a waist opening and a pair of leg openings;
a waist end edge defined by each side panel; and
a targeted elastic material in the side panels, the targeted elastic material including at least one high tension zone, at least one low tension zone, and a spacer zone between the at least one high tension zone and the waist end edge;
wherein the targeted elastic material abuts the waist end edge.

39. The disposable garment of claim 38, wherein the spacer zone comprises a second low tension zone.

40. The disposable garment of claim 38, wherein the spacer zone comprises a no tension zone.

41. The disposable garment of claim 38, wherein the spacer zone has a longitudinal length in a range from about 0.2% to about 5% of the longitudinal length of the garment.

42. The disposable garment of claim 38, wherein the spacer zone has a longitudinal length in a range from about 0.6% to about 2% of the longitudinal length of the garment.

43. The disposable garment of claim 38, wherein the spacer zone and the at least one high tension zone have a combined longitudinal length less than about 15% of the length of the garment.

44. The disposable garment of claim 38, wherein the targeted elastic material comprises a targeted elastic laminate including the low and high tension zones and the spacer zone;
the low tension zone including a plurality of elastomeric first filaments, the low tension zone having a first basis weight;
the high tension zone including a plurality of elastomeric second filaments, the high tension zone having a second basis weight higher than the first basis weight;
the laminate further including a facing layer bonded to at least a first side of the low tension zone and a first side of the high tension zone.

45. The disposable garment of claim 38, wherein the targeted elastic material comprises a targeted elastic laminate including the low and high tension zones and the spacer zone;
the low tension zone including a plurality of elastomeric first filaments, the first filaments including a first elastomeric polymer composition;
the high tension zone including a plurality of elastomeric second filaments, the second filaments including a second elastomeric polymer composition;
the laminate further including a facing material bonded to at least a first side of the low tension zone and a first side of the high tension zone.

46. A method of making garments having at least one spacer zone, comprising:
providing a continuous product assemblage;
positioning targeted elastic strips on the continuous product assemblage, the targeted elastic strips comprising a first zone and at least two second zones, the first zone corresponding to the at least one spacer zone and the first zone having lower tension than the second zones;
cutting the continuous product assemblage along a cut line located within the first zone to form a plurality of discrete garments.

47. The method of claim 46, wherein each spacer zone is located between one of the second zones and the cut line.

48. The method of claim 46, wherein the continuous product assemblage is provided in the machine direction.

49. The method of claim 46 in which the first zone coincides with an edge of the garment.

50. The method of claim 46 in which the targeted elastic strips are side panel strips.

51. A garment made according to the method of claim 46.

52. A disposable garment comprising:
a body, the body including a targeted elastic material;
at least one edge; and
at least one spacer zone aligned with the at least one edge;
wherein the targeted elastic material abuts the at least one edge.

53. The disposable garment of claim 52, wherein the body comprises a low tension zone and at least one high tension zone aligned with the at least one edge.

54. The disposable garment of claim 52, wherein the at least one spacer zone is located between the at least one high tension zone and the at least one edge.
